(19) United States
(12) Patent Application Publication
(10) Pub. No.: US 2020/0040085 A1
(43) Pub. Date: Feb. 6, 2020

(54) ANTI-HLA-DQ2.5/8 ANTIBODY AND ITS USE FOR THE TREATMENT OF CELIAC DISEASE

(71) Applicant: Chugai Seiyaku Kabushiki Kaisha, Tokyo (JP)

(72) Inventors: Yuu Okura, Singapore (SG); Noriyuki Takahashi, Singapore (SG)

(73) Assignee: Chugai Seiyaku Kabushiki Kaisha, Tokyo (JP)

(21) Appl. No.: 16/488,336

(22) PCT Filed: Feb. 26, 2018
(86) PCT No.: PCT/JP2018/007023

§ 371 Date: Aug. 23, 2019

(30) Foreign Application Priority Data
Feb. 27, 2017 (JP) 2017-035460

(51) Int. Cl. C07K 16/28; A61P 1/00; A61P 37/06; G01N 33/577

(52) U.S. Cl. CPC C07K 16/2833; A61P 1/00; A61P 37/06; G01N 33/577; C07K 2317/33; C07K 2317/77; C07K 2317/31; C07K 2317/565; G01N 2800/065; C07K 2317/92; C07K 2317/76

(57) ABSTRACT

The invention provides anti-HLA-DQ2.5/8 antibodies and methods of using the same. The antibodies of the present invention have binding activity to human HLA-DQ2.5/8 and monkey MHC-DQ, but substantially no binding activity to HLA-DR, HLA-DP, or a complex of the invariant chain (CD74) and HLA-DQ2.5/8. The antibodies bind to HLA-DQ2.5/8 in the presence of a gluten peptide such as gliadin, i.e., bind to HLA-DQ2.5/8 forming a complex with the gluten peptide. The antibodies have neutralizing activity against the binding between HLA-DQ2.5/8 and TCR, and thus block the interaction between HLA-DQ2.5/8 and an HLA-DQ2.5/8-restricted CD4+ T cell. The antibodies do not undergo rapid internalization mediated by the invariant chain. These characteristics are particularly useful for treating celiac disease. Thus, the present invention also provides a method of treating celiac disease using the antibodies of the present invention.

### TECHNICAL FIELD

The present invention relates to anti-HLA-DQ2.5/8 antibodies and methods of using the same.

### BACKGROUND ART

Celiac (coeliac) disease is an autoimmune disorder in which the ingestion of gluten causes damage to the small intestine in genetically-sensitive patients (NPL 1 to 5). About 1% of the Western population, i.e., 8 million people in the United States and the European Union are thought to suffer from celiac disease; however, no remarkable therapeutic advances have been achieved since the disease was recognized in 1940s.

Human leukemia antigens (HLAs) belonging to Major Histocompatibility Complex (MHC) class II include HLA-DR, HLA-DP and HLA-DQ such as the HLA-DQ2.5 and/or HLA-DQ8 (hereinafter referred to as “HLA-DQ2.5/8”) isoforms, which form heterodimers composed of alpha (alpha) and beta (beta) chains on the cell surface. A majority (>90%) of the celiac disease patients have an HLA-DQ2.5 haplotype allele, and most of the remaining patients possess an HLA-DQ8 allele (NPL 6). These isoforms are thought to have stronger affinity towards a gluten peptide. As with other isoforms, the HLA-DQ2.5 and HLA-DQ8 molecules present processed antigens derived from exogenous sources to a T cell receptor (TCR) on T cells. As a result of digestion of gluten-rich food such as bread in celiac disease patients, immunogenic gluten peptides such as gliadin peptides are formed (NPL 2). The peptides are transported through the small intestine epithelium into lamina propria and deamidated by tissue transglutaminase such as transglutaminase 2 (TG2). The deamidated gliadin peptides are processed by antigen-presenting cells (APCs) which load them on the HLA-DQ2.5/8 molecules. The loaded peptides are presented to HLA-DQ2.5/8-restricted T cells, and activate innate and adaptive immune responses. This causes inflammatory injury of the small intestinal mucosa and symptoms including various types of gastrointestinal disturbance, nutritional deficiencies, and systemic symptoms.

The currently practicable treatment of celiac disease is lifelong adherence to a glutenfree diet (GFD). However, in reality, it is difficult to completely eliminate gluten exposure even with GFD. The tolerable gluten dose for these patients is only about 10 to 50 mg/day (NPL 10). Cross contamination can widely occur in GFD production, and a trace amount of gluten can cause celiac disease symptoms even in patients with good compliance to GFD. In the presence of such a risk of unintentional gluten exposure, there is a need for adjunctive therapy to GFD.

### CITATION LIST

### Non Patent Literature

[NPL 1] N Engl J Med 2007; 357:1731-1743
[NPL 2] J Biomed Sci. 2012; 19(1): 88
[NPL 3] N Engl J Med 2003; 348:2517-2524
[NPL 4] Gut 2003; 52:960-965
[NPL 5] Dig Dis Sci 2004; 49:1479-1484
[NPL 6] Gastroenterology 2011; 141:610-620
[NPL 7] Gastroenterology 2014; 146:1649-58
[NPL 8] Nutrients 2013 Oct. 5(10): 3975-3992
[NPL 9] J Clin Invest. 2007; 117(1):41-49
[NPL 10] Am J Clin Nutr 2007; 85: 160-6

### SUMMARY OF INVENTION

### Technical Problem

Under the above-mentioned circumstances with the need for adjunctive therapy, the present invention provides anti-HLA-DQ2.5/8 antibodies and methods of using the same.

### Solution to Problem

In certain embodiments, the anti-HLA-DQ2.5/8 antibody of the present invention (hereinafter also referred to as “the antibody of the present invention”) has binding activity to human HLA-DQ2.5/8 and monkey MHC-DQ.

In certain embodiments, the antibody of the present invention has substantially no binding activity to HLA-DR or HLA-DP.

In certain embodiments, the antibody of the present invention has substantially no binding activity to a complex of the invariant chain and HLA-DQ2.5/8.

In certain embodiments, the antibody of the present invention binds to HLA-DQ2.5/8 in the presence of a gluten peptide such as gliadin.

In certain embodiments, the antibody of the present invention has neutralizing activity against the binding between HLA-DQ2.5/8 and TCR.

In certain embodiments, the antibody of the present invention blocks the interaction between HLA-DQ2.5/8 and an HLA-DQ2.5/8-restricted CD4+ T cell.

In certain embodiments, the antibody of the present invention does not undergo cell internalization with the invariant chain (i.e., invariant chain-mediated rapid cell internalization).

In certain embodiments, the antibody of the present invention has pH dependent generation or optimization.

In certain embodiments, the antibody of the present invention is a humanized antibody. In certain embodiments, the antibody of the present invention has specific heavy-chain complementarity determining regions (HCDRs).

In certain embodiments, the antibody of the present invention has specific light-chain complementarity determining regions (LCDRs).

In certain embodiments, the present invention provides an antibody that binds to the same HLA-DQ2.5/8 epitope bound by the antibody that has the specific HCDRs and LCDRss.

In certain embodiments, the present invention provides an antibody that competes for HLA-DQ2.5/8 binding with the antibody that has the specific HCDRs and LCDRs. In certain embodiments, the present invention provides a method for treating celiac disease, comprising administering to a patient a therapeutically effective amount of the antibody of the present invention.

In certain embodiments, the antibody of the present invention is for use in treating celiac disease.

In certain embodiments, the present invention provides a pharmaceutical composition for use in treating celiac disease, which comprises the antibody of the present invention and a pharmaceutically acceptable carrier.

In certain embodiments, the present invention provides a method of screening for an anti-HLA-DQ2.5/8 antibody, which comprises testing whether the antibody has binding activity to an antigen(s) of interest and selecting the antibody that has binding activity to the antigen(s) of interest; testing whether the antibody has specific binding activity to an antigen(s) of no interest and selecting the antibody that has no specific binding activity to the antigen(s) of no interest.

In certain embodiments, the above method further comprises: testing whether the antibody has neutralizing activity against the binding between HLA-DQ2.5/8 and TCR; and selecting the antibody that has the neutralizing activity.

In certain embodiments, the above method further comprises: testing whether the antibody binds to HLA-DQ2.5/8 in the presence of a gluten peptide such as gliadin; and selecting the antibody that binds to HLA-DQ2.5/8 in the presence of the gluten peptide.

In certain embodiments, the present invention provides a method of preparing a pharmaceutical formulation for use in treating celiac disease, which comprises mixing a pharmaceutically acceptable carrier and the antibody of the present invention selected by (a step of) the above screening method.

More specifically, the present invention provides the following.

[1] An anti-HLA-DQ2.5/8 antibody, wherein

a) the antibody has binding activity to HLA-DQ2.5 and HLA-DQ8,

b) the antibody has substantially no binding activity to HLA-DR or HLA-DP, and

c) the antibody has substantially no binding activity to a complex of invariant chain and HLA-DQ2.5.

[2] The antibody of [1], wherein the antibody has binding activity to HLA-DQ2.5 and HLA-DQ8 in the presence of a gluten peptide.

[3] The antibody of [2], wherein the gluten peptide is gliadin.

[4] The antibody of [1], wherein the antibody has binding activity to monkey MHC-DQ.

[5] The antibody of [1], wherein the antibody blocks the interaction between HLA-DQ2.5 and an HLA-DQ2.5 restricted CD4+ T cell, and the antibody blocks the interaction between HLA-DQ8 and an HLA-DQ8 restricted CD4+ T cell.

[6] An anti-HLA-DQ2.5/8 antibody, wherein the antibody binds to HLA-DQ2.5 and HLA-DQ8 in the presence of a gluten peptide without cell internalization mediated by an invariant chain.

[7] The antibody of [1], which is any one of (1) to (16) below:

(1) an antibody comprising the HCDR1 sequence of SEQ ID NO: 9, the HCDR2 sequence of SEQ ID NO: 17, the HCDR3 sequence of SEQ ID NO: 25, the LCDR1 sequence of SEQ ID NO: 41, the LCDR2 sequence of SEQ ID NO: 49, and the LCDR3 sequence of SEQ ID NO: 57;

(2) an antibody comprising the VH sequence of SEQ ID NO: 1 and the VL sequence of SEQ ID NO: 33;

(3) an antibody comprising the HCDR1 sequence of SEQ ID NO: 10, the HCDR2 sequence of SEQ ID NO: 18, the HCDR3 sequence of SEQ ID NO: 26, the LCDR1 sequence of SEQ ID NO: 42, the LCDR2 sequence of SEQ ID NO: 50, and the LCDR3 sequence of SEQ ID NO: 58;

(4) an antibody comprising the VH sequence of SEQ ID NO: 2 and the VL sequence of SEQ ID NO: 34;

(5) an antibody comprising the HCDR1 sequence of SEQ ID NO: 11, the HCDR2 sequence of SEQ ID NO: 19, the HCDR3 sequence of SEQ ID NO: 27, the LCDR1 sequence of SEQ ID NO: 43, the LCDR2 sequence of SEQ ID NO: 51, and the LCDR3 sequence of SEQ ID NO: 59;

(6) an antibody comprising the VH sequence of SEQ ID NO: 3 and the VL sequence of SEQ ID NO: 35;

(7) an antibody comprising the HCDR1 sequence of SEQ ID NO: 12, the HCDR2 sequence of SEQ ID NO: 20, the HCDR3 sequence of SEQ ID NO: 28, the LCDR1 sequence of SEQ ID NO: 44, the LCDR2 sequence of SEQ ID NO: 52, and the LCDR3 sequence of SEQ ID NO: 60;

(8) an antibody comprising the VH sequence of SEQ ID NO: 4 and the VL sequence of SEQ ID NO: 36;

(9) an antibody comprising the HCDR1 sequence of SEQ ID NO: 13, the HCDR2 sequence of SEQ ID NO: 21, the HCDR3 sequence of SEQ ID NO: 29, the LCDR1 sequence of SEQ ID NO: 45, the LCDR2 sequence of SEQ ID NO: 53, and the LCDR3 sequence of SEQ ID NO: 61;

(10) an antibody comprising the VH sequence of SEQ ID NO: 5 and the VL sequence of SEQ ID NO: 37;

(11) an antibody comprising the HCDR1 sequence of SEQ ID NO: 14, the HCDR2 sequence of SEQ ID NO: 22, the HCDR3 sequence of SEQ ID NO: 30, the LCDR1 sequence of SEQ ID NO: 46, the LCDR2 sequence of SEQ ID NO: 54, and the LCDR3 sequence of SEQ ID NO: 62;

(12) an antibody comprising the VH sequence of SEQ ID NO: 6 and the VL sequence of SEQ ID NO: 38;

(13) an antibody comprising the HCDR1 sequence of SEQ ID NO: 16, the HCDR2 sequence of SEQ ID NO: 24, the HCDR3 sequence of SEQ ID NO: 32, the LCDR1 sequence of SEQ ID NO: 48, the LCDR2 sequence of SEQ ID NO: 56, and the LCDR3 sequence of SEQ ID NO: 64;

(14) an antibody comprising the VH sequence of SEQ ID NO: 8 and the VL sequence of SEQ ID NO: 40;

(15) an antibody that binds to the same HLA-DQ2.5 epitope and HLA-DQ8 epitope bound by the antibody of any one of (1) to (14);

(16) an antibody that competes for binding of HLA-DQ2.5 and HLA-DQ8 with the antibody of any one of (1) to (14).

[8] A method for treating celiac disease, comprising administering to a patient a therapeutically effective amount of the antibody of any one of [1] to [7].

[9] The antibody of any one of [1] to [7] for use in treating celiac disease.

[10] A pharmaceutical composition for use in treating celiac disease, which comprises the antibody of any one of [1] to [7] and a pharmaceutically acceptable carrier.

[11] A method of screening for an anti-HLA-DQ2.5/8 antibody, which comprises:

(a) testing whether an antibody has binding activity to HLA-DQ2.5 and HLA-DQ8; and selecting an antibody that has binding activity to HLA-DQ2.5 and HLA-DQ8;

(b) testing whether an antibody has a specific binding activity to HLA-DR or HLA-DP; and selecting an antibody that has substantially no specific binding activity to HLA-DR or HLA-DP;

(c) testing whether an antibody has a specific binding activity to a complex of the invariant chain and HLA-DQ2.5; and selecting an antibody that has substantially no specific binding activity to the complex of the invariant chain and HLA-DQ2.5.

[12] The method of [11], which further comprises: testing whether an antibody has binding activity to HLA-DQ2.5 and HLA-DQ8 in the presence of a gluten peptide; and selecting an antibody that has binding activity to HLA-DQ2.5 and HLA-DQ8 in the presence of the gluten peptide.

[13] The method of [11], which further comprises: testing whether an antibody has binding activity to monkey MHC-DQ; and selecting an antibody that has binding activity to monkey MHC-DQ.

[14] The method of [11], which further comprises: testing whether an antibody has neutralizing activity against the binding between HLA-DQ2.5 and TCR and the binding between HLA-DQ8 and TCR; and selecting an antibody that has the neutralizing activity.

[15] A method of preparing a pharmaceutical formulation for use in treating celiac disease, which comprises mixing a pharmaceutically acceptable carrier and the antibody selected by the method of [11].

### BRIEF DESCRIPTION OF DRAWINGS

FIG. 1 shows FACS results of the binding of the antibodies to HLA-DQ2.5 (Example 4.1).

FIG. 2 shows FACS results of the binding of the antibodies to HLA-DQ8 (Example 4.1).

FIG. 3 shows FACS results of the binding of the antibodies to HLA-DR (Example 4.1).

FIG. 4 shows FACS results of the binding of the antibodies to HLA-DP (Example 4.1).

FIG. 5 shows FACS results of the binding of the antibodies to cynomolgus monkey MHC DQ (Example 4.1).

FIG. 6 shows the neutralizing activity of the antibodies (Example 4.3).

FIG. 7 shows the binding of the antibodies to the HLA-DQ2.5/invariant chain complex (Example 4.4).

FIG. 8 shows the rate of antibody internalization mediated by the HLA-DQ/invariant chain complex (Example 4.5).

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-HLA-DQ2.5/8 antibody” and “an antibody that binds to HLA-DQ2.5/8” refer to an antibody that is capable of binding HLA-DQ2.5 and/or HLA-DQ8 (herein referred to as “HLA-DQ2.5/8”) with sufficient affinity such that the antibody is useful as a diagnostic and/or therapeutic agent in targeting HLA-DQ2.5/8. In one embodiment, the extent of binding of an anti-HLA-DQ2.5/8 antibody to an unrelated, non-HLA-DQ2.5/8 protein is less than about 10% of the binding of the antibody to HLA-DQ2.5/8 as measured, e.g., by a radioimmunoassay (RIA). In certain embodiments, an antibody that binds to HLA-DQ2.5/8 has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³M, e.g., from 10⁻⁹M to 10⁻¹³M). In certain embodiments, an anti-HLA-DQ2.5/8 antibody binds to an epitope of HLA-DQ2.5/8 that is conserved among HLA-DQ2.5/8 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, celiac disease, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobulinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

The term “celiac (coeliac) disease” refers to a hereditary autoimmune disease caused by damages in the small intestine upon ingenstion of gluten contained in food. Symptoms of celiac disease include, but not limited to, gastrointestinal disturbance such as abdominal pain, diarrhea, and gastroesophageal reflux, vitamin deficiency, mineral deficiency, central nervous system (CNS) symptoms such as fatigue and anxiety depression, bone symptoms such as osteomalacia and osteoporosis, skin symptoms such as skin inflammation, blood symptoms such as anemia and lymphocytopenia, and other symptoms such as infertility, hypogonadism, and children's failure to thrive and short stature.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

Herein, the term “gluten” collectively refers to a composite of storage proteins called prolamins found in wheat and other related grains. In the gut lumen, gluten is degraded into so-called gluten peptides. Gluten peptides include, but are not limited to, gliadin from wheat, hordein from barley, and secalin from rye.

The phrase “having substantially no binding activity”, as used herein, refers to activity of an antibody to bind to an antigen of no interest at a level of binding that includes non-specific or background binding but does not include specific binding. In other words, such an antibody “has no specific/significant binding activity” towards the antigen of no interest. The specificity can be measured by any methods mentioned in this specification or known in the art. The above-mentioned level of non-specific or background binding may be zero, or may not be zero but near zero, or may be very low enough to be technically neglected by those skilled in the art. For example, when a skilled person cannot detect or observe any significant (or relatively strong) signal for binding between the antibody and the antigen of no interest in a suitable binding assay, it can be said that the antibody has “substantially no binding activity” or “no specific/significant binding activity” towards the antigen of no interest. Alternatively, “have substantially no binding activity” or “have no specific/significant binding activity” can be rephrased as “do/does not specifically/significantly/substantially bind” (to the antigen of no interest). Sometimes, the phrase “having no binding activity” has substabtially the same meaning as the phrase “having substantially no binding activity” or “having no specific/significant binding activity” in the art.

The phrase “having undergone pH-dependent generation/optimization” means that the antibody of interest has been prepared or modified to confer any pH-dependent characteristics. An example of such characteristics is pH-dependent binding of an antibody of the present invention to HLA-DQ2.5/8.

Herein, “HLA-DR/DP” and “HLA-DQ2.5/8” respectively mean “HLA-DR and/or HLA-DP” and “HLA-DQ2.5 and/or HLA-DQ8”. These HLAs are MHC class II molecules encoded by the corresponding haplotype alleles on the MHC class II locus in human. “HLA-DQ” collectively refers to HLA-DQ isoforms including HLA-DQ2.5 and HLA-DQ8. Similarly, “HLA-DR (DP)” refers to HLA-DR (DP) isoforms.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

In one embodiment, HVR residues comprise those identified in the specification.

Unless otherwise indicated, HVR residues and other residues in the variable domain (e.g., FR residues) are numbered herein according to Kabat et al., supra.

An “immunoconjugate” is an antibody conjugated to one or more heterologous molecule(s).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

Herein, “(the) invariant chain” refers to a protein encoded by a gene for human CD74 (GenBank accession No. NM_001025159). Thus, “invariant chain” is also called “CD74” or “CD74/invariant chain”. The invariant chain forms a complex with an MHC class II molecule such as HLA-DQ2.5/8, and this complex can be located on the membrane of the endoplasmic reticulum or the endosome, or the plasma membrane of an MHC class II-expressing cell. The term “invariant chain (76-295)” refers to the partial peptide consisting of the amino acid residues from positions 76 to 295 of the invariant chain according to GenBank accession No. NM_001025159.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-HLA-DQ2.5/8 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

Herein, “MHC-DQ” refers to an MHC class II (HLA-DQ) counterpart in monkey. In the present invention, MHC-DQ used in the Examples is a preferable molecule. Without any limitations, “monkey” includes cynomolgus monkey or crab-eating macque (Macaca fascicularis), rhesus macaque (Macaca mulatta), and other genus/species of monkeys having MHC-DQ.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (kappa) and lambda (lambda), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “HLA-DQ2.5/8,” as used herein, refers to any native HLA-DQ2.5/8 from any vertebrate source, including mammals such as primates (e.g. humans) and rodents (e.g., mice and rats), unless otherwise indicated. The term encompasses “full-length” unprocessed HLA-DQ2.5/8 as well as any form of HLA-DQ2.5/8 that results from processing in the cell. The term also encompasses naturally occurring variants of HLA-DQ2.5/8, e.g., splice variants or allelic variants. The amino acid sequence of exemplary HLA-DQ2.5/8 is publicly available in Research Collaboratory for Structural Bioinformatics (RCSB) Protein Data Bank (PDB) accession code 4OZG (DQ2.5) or 4GG6 (DQ8).

Herein, “TCR” means “T-cell receptor” which is a membrane protein located on the surface of T cells (such as HLA-DQ2.5/8-restricted CD4+ T cells), and recognizes an antigen fragment (such as a gluten peptide) presented on MHC molecules including HLA-DQ2.5/8.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In one aspect, the invention is based, in part, on the binding of an anti-HLA-DQ2.5/8 antibody to HLA-DQ2.5/8 that presents a gluten peptide to T cells. In certain embodiments, antibodies that bind to HLA-DQ2.5/8 are provided. Antibodies of the invention are useful, e.g., for the diagnosis or treatment of celiac disease.

A. Exemplary Anti-HLA-DQ2.5/8 Antibodies

In one aspect, the invention provides isolated antibodies that bind to HLA-DQ2.5/8. In certain embodiments, the anti-HLA-DQ2.5/8 antibody (“the antibody”) has the functions/characteristics below.

The antibody has binding activity to HLA-DQ2.5/8. In other words, the antibody binds to HLA-DQ2.5/8. More preferably, the antibody has specific binding activity to HLA-DQ2.5/8. That is, the antibody specifically binds to HLA-DQ2.5/8.

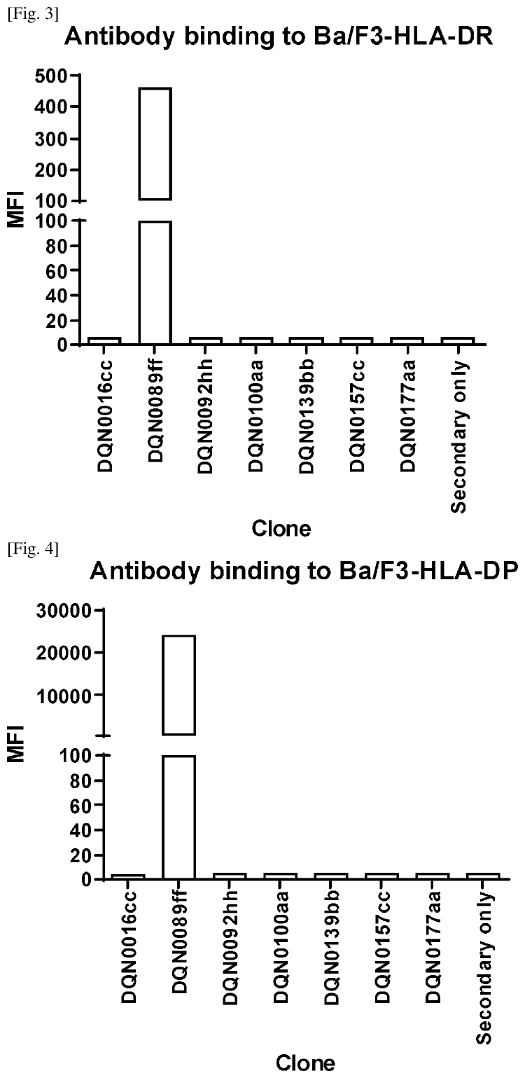

The antibody has substantially no binding activity to HLA-DR/DP, i.e., the antibody does not substantially bind to HLA-DR/DP. In other words, the antibody has no specific binding activity to HLA-DR/DP or no significant binding activity to HLA-DR/DP. That is, the antibody does not specifically bind to HLA-DR/DP or significantly bind to HLA-DR/DP. To prevent any substantial inhibitory effects on these non-target MHC class II molecules, these characteristics are preferable.
The feature of the “substantially no binding activity” can be defined, for example, as described in the FACS results of FIGS. 3 to 5. The antibody having “substantially no binding activity” to a specific antigen has an MFI (Mean Fluorescence Intensity) value that is 200% or less, preferably 150% or less of the MFI value of the negative control (Secondary only) under the measurement conditions of Example 4.1.

The antibody has binding activity to monkey MHC-DQ. In other words, the antibody binds to monkey MHC-DQ. More preferably, the antibody has a specific binding activity to monkey MHC-DQ. That is, the antibody specifically binds to monkey MHC-DQ. The monkey MHC-DQ is preferably cynomolgus monkey MHC DQ. The binding to monkey MHC-DQ which is highly homologous to the human counterpart is more preferable in that the antibody is expected to have a strong affinity to the human counterpart.

The antibody has binding activity to HLA-DQ2.5/8 in the presence of a gluten peptide, i.e., the antibody binds to HLA-DQ2.5/8 in the presence of a gluten peptide. More preferably, the antibody has a specific binding activity to HLA-DQ2.5/8 in the presence of a gluten peptide, i.e., the antibody specifically binds to HLA-DQ2.5/8 in the presence of a gluten peptide. In other words, the antibody (specifically) binds to HLA-DQ2.5/8 bound by a gluten peptide, i.e., a complex of HLA-DQ2.5/8 and a gluten peptide. The gluten peptide is preferably gliadin. The gluten peptide (gliadin)-dependent binding is preferable for use in treating celiac disease.
Anti-HLA-DQ2.5/8 antibodies of the invention have a dissociation constant (Kd) of 1×10⁻⁸M or less, preferably 7×10⁻⁹ M or less for binding to HLA-DQ2.5/gliadin peptide, and/or such antibodies have a dissociation constant (Kd) of 2×10⁻⁷ M or less, preferably 5×10⁻⁸M or less, more preferably 1×10⁻⁸M or less for binding to HLA-DQ8/gliadin peptide under the measurement conditions described in Example 4.2.

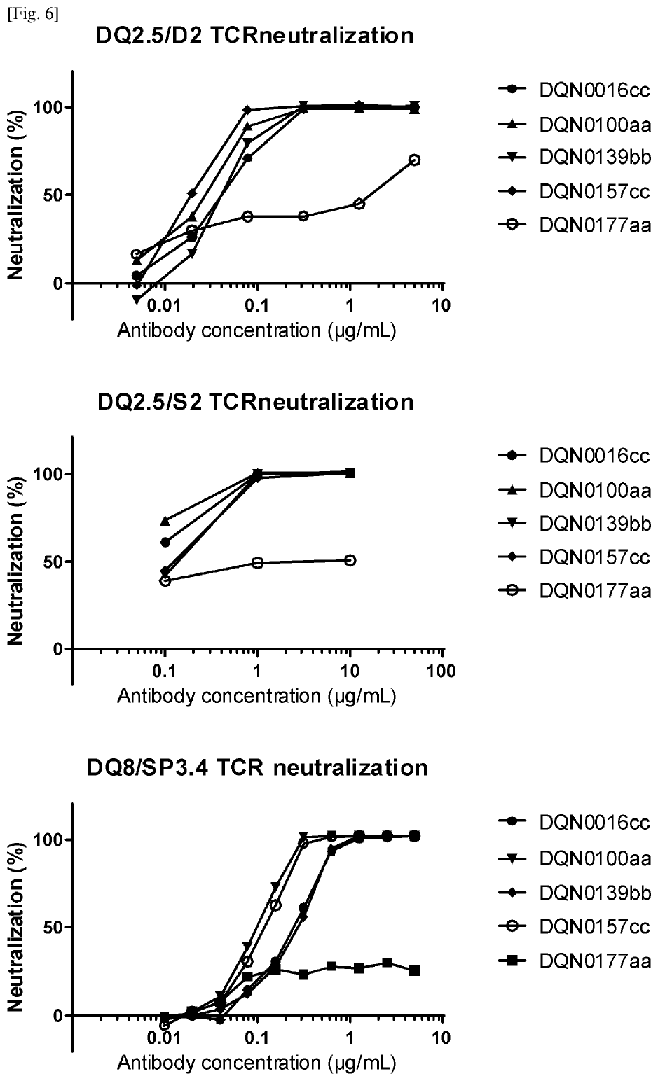

The antibody has neutralizing activity against the binding between HLA-DQ2.5/8 and TCR. In other words, the antibody blocks the binding between HLA-DQ2.5/8 and TCR. More preferably, such binding occurs in the presence of a gluten peptide, i.e., when HLA-DQ2.5/8 is bound by a gluten peptide, or forms a complex with a gluten peptide. The gluten peptide is preferably gliadin. The antibody blocks the interaction between HLA-DQ2.5/8 and an HLA-DQ2.5/8-restricted CD4+ T cell. The blocking can be achieved by the above-mentioned binding between HLA-DQ2.5/8 and TCR.
The feature of the “neutralizing activity” can be defined, for example, as described in FIG. 6. The antibody having the “neutralizing activity” can neutralize the binding between HLA-DQ2.5 and TCR for 95% or more, preferably 97% or more, more preferably 99% or more by antibody concentration of 1 micro g/mL under the measurement conditions described in Example 4.3. Additionally, the antibody having the “neutralizing activity” can neutralize the binding between HLA-DQ8 and TCR for 95% or more, preferably 97% or more, more preferably 99% or more by antibody concentration of 1 micro g/mL under the measurement conditions described in Example 4.3.

The antibody has substantially no binding activity to (does not substantially bind to) the invariant chain (CD74). In other words, the antibody has no specific/significant binding activity to (does not specifically/significantly bind to) the invariant chain. The HLA-DQ molecules are localized on the cell surface with or without the invariant chain. When HLA-DQ forms a complex with the invariant chain, the complex on the cell surface is rapidly internalized into the endosome (rapid cell internalization called “rapid internalization”). In the endosome, the invariant chain is degraded by protease, and free HLA-DQ is loaded with a peptide such as a gluten peptide. The HLA-DQ/peptide complex is transferred to the cell surface, and then recognized by TCR on T cells. This can cause celiac disease. The complex without the invariant chain is slowly internalized into the endosome (slow cell internalization called “slow internalization”). The absence of binding to the invariant chain is preferable since the antibody is less susceptible to rapid internalization which can cause the antibody to be quickly transferred to the endosome with the invariant chain and degraded (see also Example 4.4).

The antibody has substantially no binding activity to (does not substantially bind to) a complex of the invariant chain and HLA-DQ2.5/8. In other words, the antibody has no specific/significant binding activity to (does not specifically/significantly bond to) the complex of the invariant chain and HLA-DQ2.5/8. That is, the antibody does not undergo antibody internalization (“rapid internalization”) mediated by the invariant chain. These characteristics can be achieved by the above-mentioned absence of binding to the invariant chain.

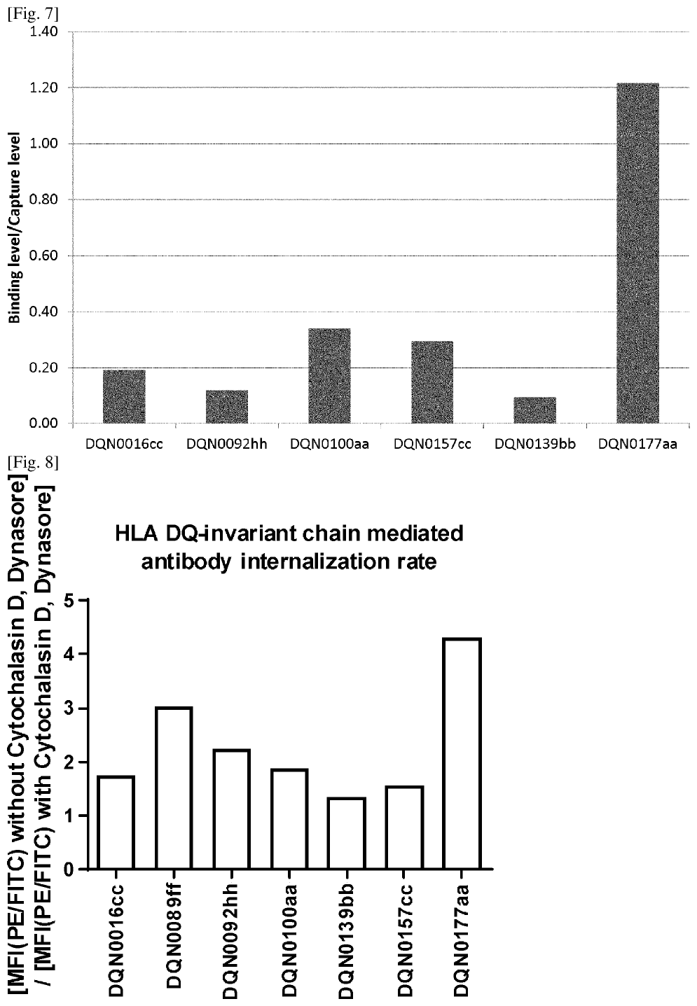

The feature of the “substantially no binding activity” can be defined, for example, as described in FIG. 7. An anti-HLA-DQ2.5/8 antibody having “substantially no binding activity” to a specific antigen (i.e., HLA-DQ2.5/8-invariant chain) has a binding/capture value of 0.4 or less, i.e., level of binding of an anti-HLA-DQ2.5/8 antibody to human HLA-DQ2.5-invariant chain/level of the anti-HLA-DQ2.5/8 antibody being captured, under the measurement conditions described in Example 4.4.

In one aspect, the invention provides an anti-HLA-DQ2.5/8 antibody comprising at least one, two, three, four, five, or six HVRs (CDRs) selected from (a) HVR-H1 (HCDR1) comprising the amino acid sequence of any one of SEQ ID NOs: 9 to 16; (b) HVR-H2 (HCDR2) comprising the amino acid sequence of any one of SEQ ID NOs: 17 to 24; (c) HVR-H3 (HCDR3) comprising the amino acid sequence of any one of SEQ ID NOs: 25 to 32; (d) HVR-L1 (LCDR1) comprising the amino acid sequence of any one of SEQ ID NOs: 41 to 48; (e) HVR-L2 (LCDR2) comprising the amino acid sequence of any one of SEQ ID NOs: 49 to 56; and (f) HVR-L3 (LCDR3) comprising the amino acid sequence of any one of SEQ ID NOs: 57 to 64.

In one aspect, the invention provides an antibody comprising at least one or two, or all three of the VH HVR (HCDR) sequences selected from (a) HVR-H1 (HCDR1) comprising the amino acid sequence of any one of SEQ ID NOs: 9 to 16; (b) HVR-H2 (HCDR2) comprising the amino acid sequence of any one of SEQ ID NOs: 17 to 24; and (c) HVR-H3 (HCDR3) comprising the amino acid sequence of any one of SEQ ID NOs: 25 to 32.

In another aspect, the invention provides an antibody comprising at least one or two, or all three of the VL HVR (LCDR) sequences selected from (a) HVR-L1 (LCDR1) comprising the amino acid sequence of any one of SEQ ID NOs: 41 to 48; (b) HVR-L2 (LCDR2) comprising the amino acid sequence of any one of SEQ ID NOs: 49 to 56; and (c) HVR-L3 (LCDR3) comprising the amino acid sequence of any one of SEQ ID NOs: 57 to 64

In another aspect, an antibody of the invention comprises (a) a VH domain comprising at least one or two, or all three of the VH HVR (HCDR) sequences selected from (i) HVR-H1 (HCDR1) comprising the amino acid sequence of any one of SEQ ID NOs: 9 to 16, (ii) HVR-H2 (HCDR2) comprising the amino acid sequence of any one of SEQ ID NOs: 17 to 24, and (iii) HVR-H3 (HCDR3) comprising an amino acid sequence of any one of SEQ ID NOs: 25 to 32; and (b) a VL domain comprising at least one or two, or all three of the VL HVR (LCDR) sequences selected from (i) HVR-L1 (LCDR1) comprising the amino acid sequence of any one of SEQ ID NOs: 41 to 48, (ii) HVR-L2 (LCDR2) comprising the amino acid sequence of any one of SEQ ID NOs: 49 to 56, and (c) HVR-L3 (LCDR3) comprising the amino acid sequence of any one of SEQ ID NOs: 57 to 64.

In another aspect, the invention provides an antibody comprising (a) HVR-H1 (HCDR1) comprising the amino acid sequence of any one of SEQ ID NOs: 9 to 16; (b) HVR-H2 (HCDR2) comprising the amino acid sequence of any one of SEQ ID NOs: 17 to 24; (c) HVR-H3 (HCDR3) comprising the amino acid sequence of any one of SEQ ID NOs: 25 to 32; (d) HVR-L1 (LCDR1) comprising the amino acid sequence of any one of SEQ ID NOs: 41 to 48; (e) HVR-L2 (LCDR2) comprising the amino acid sequence of any one of SEQ ID NOs:49 to 56; and (f) HVR-L3 (LCDR3) comprising an amino acid sequence selected from any one of SEQ ID NOs: 57 to 64.

In another aspect, the sequence ID numbers (SEQ ID NOs) of the VH, VL, HCDR1, HCDR2, HCDR3, LCDR1, LCDR2, and LCDR3 sequences for the following antibodies of the present invention are:
SEQ ID NOs: 1, 33, 9, 17, 25, 41, 49, and 57 for the DQN0016 antibody, respectively;
SEQ ID NOs: 2, 34, 10, 18, 26, 42, 50, and 58 for the DQN0089 antibody, respectively;
SEQ ID NOs: 3, 35, 11, 19, 27, 43, 51, and 59 for the DQN0092 antibody, respectively;
SEQ ID NOs: 4, 36, 12, 20, 28, 44, 52, and 60 for the DQN0100 antibody, respectively;
SEQ ID NOs: 5, 37, 13, 21, 29, 45, 53, and 61 for the DQN0157 antibody, respectively;
SEQ ID NOs: 6, 38, 14, 22, 30, 46, 54, and 62 for the DQN0139 antibody, respectively;
and
SEQ ID NOs: 8, 40, 16, 24, 32, 48, 56, and 64 for the DQN0177 antibody, respectively.

In certain embodiments, any one or more amino acids of an anti-HLA-DQ2.5/8 antibody as provided above are substituted in any of the HVR positions.

In certain embodiments, the substitutions are conservative substitutions, as provided herein.

In any of the above embodiments, an anti-HLA-DQ2.5/8 antibody is humanized. In one embodiment, an anti-HLA-DQ2.5/8 antibody comprises HVRs as in any of the above embodiments, and further comprises an acceptor human framework, e.g., a human immunoglobulin framework or a human consensus framework. In another embodiment, an anti-HLA-DQ2.5/8 antibody comprises HVRs as in any of the above embodiments, and further comprises the FR1, FR2, FR3, or FR4 sequence shown in Table 1 below.

In another aspect, an anti-HLA-DQ2.5/8 antibody comprises a heavy-chain variable domain (VH) sequence having at least 90%, 91%, 92%, 93%, 94%, 95%, 96%, 97%, 98%, 99%, or 100% sequence identity to the amino acid sequence of any one of SEQ ID NOs: 1 to 8. In certain embodiments, a VH sequence having at least 90%, 91%, 92%, 93%, 94%, 95%, 96%, 97%, 98%, or 99% identity comprises substitutions (e.g., conservative substitutions), insertions, or deletions relative to the reference sequence, but an anti-HLA-DQ2.5/8 antibody comprising that sequence retains the ability to bind to HLA-DQ2.5/8. In certain embodiments, a total of 1 to 10 amino acids have been substituted, inserted and/or deleted in any one of SEQ ID NOs: 1 to 8. In certain embodiments, substitutions, insertions, or deletions occur in regions outside the HVRs (i.e., in the FRs). Optionally, the anti-HLA-DQ2.5/8 antibody comprises the VH sequence of any one of SEQ ID NOs: 1 to 8 or a sequence comprising a post-translational modification thereof. In a particular embodiment, the VH comprises one, two or three HVRs selected from: (a) HVR-H1 comprising the amino acid sequence of any one of SEQ ID NOs: 9 to 16, (b) HVR-H2 comprising the amino acid sequence of any one of SEQ ID NOs:17 to 24, and (c) HVR-H3 comprising the amino acid sequence of any one of SEQ ID NOs: 25 to 32. Post-translational modifications include but are not limited to a modification of glutamine or glutamate at the N terminus of the heavy chain or light chain to pyroglutamic acid by pyroglutamylation.

In another aspect, an anti-HLA-DQ2.5/8 antibody is provided, wherein the antibody comprises a light-chain variable domain (VL) having at least 90%, 91%, 92%, 93%, 94%, 95%, 96%, 97%, 98%, 99%, or 100% sequence identity to the amino acid sequence of any one of SEQ ID NOs: 33 to 40. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-HLA-DQ2.5/8 antibody comprising that sequence retains the ability to bind to HLA-DQ2.5/8. In certain embodiments, a total of 1 to 10 amino acids have been substituted, inserted and/or deleted in any one of SEQ ID NOs: 33 to 40. In certain embodiments, the substitutions, insertions, or deletions occur in regions outside the HVRs (i.e., in the FRs). Optionally, the antiHLA-DQ2.5/8 antibody comprises the VL sequence of any one of SEQ ID NOs: 33 to 40 or a sequence comprising a post-translational modification thereof. In a particular embodiment, the VL comprises one, two or three HVRs selected from (a) HVR-L1 comprising the amino acid sequence of any one of SEQ ID NOs: 41 to 48; (b) HVR-L2 comprising the amino acid sequence of any one of SEQ ID NOs: 49 to 56; and (c) HVR-L3 comprising the amino acid sequence of any one of SEQ ID NOs: 57 to 64. Post-translational modifications include but are not limited to a modification of glutamine or glutamate at the N terminus of the heavy chain or light chain to pyroglutamic acid by pyroglutamylation.

In another aspect, an anti-HLA-DQ2.5/8 antibody is provided, wherein the antibody comprises a VH as in any of the embodiments provided above, and a VL as in any of the embodiments provided above. In one embodiment, the antibody comprises the VH sequence of any one of SEQ ID NOs: 1 to 8 or a sequence comprising a post-translational modification thereof, and the VL sequence of any one of SEQ ID NOs: 33 to 40 or a sequence comprising a post-translational modification thereof. Post-translational modifications include but are not limited to a modification of glutamine or glutamate at the N terminus of the heavy chain or light chain to pyroglutamic acid by pyroglutamylation.

In a further aspect, the invention provides an antibody that binds to the same epitope as an anti-HLA-DQ2.5/8 antibody provided herein. For example, in certain embodiments, an antibody is provided that binds to the same epitope as any of the above-mentioned antbodies, DQN0016, DQN0089, DQN0092, DQN0100, DQN0157, DQN0139, and DQN 0177. In certain embodiments, an antibody is provided that binds to an epitope within a fragment of HLA-DQ2.5/8 consisting of about 8 to 17 amino acids.

In a further aspect of the invention, an anti-HLA-DQ2.5/8 antibody according to any of the above embodiments is a monoclonal antibody, including a chimeric, humanized or human antibody. In one embodiment, an anti-HLA-DQ2.5/8 antibody is an antibody fragment, e.g., a Fv, Fab, Fab′, scFv, diabody, or F(ab′)₂ fragment. In another embodiment, the antibody is a full-length antibody, e.g., an intact IgG1 antibody or other antibody class or isotype as defined herein.

In a further aspect, an anti-HLA-DQ2.5/8 antibody according to any of the above embodiments may incorporate any of the features described in Sections 1-7 below, whether singly or in combination:

1. Antibody Affinity

In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻¹³ M to 10¹³M, e.g., from 10⁻⁹ M to 10⁻¹³M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, N.J.) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and Nhydroxysuccinimide (NHS) according to the supplier's instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm bandpass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Da11′Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB (registered trademark) technology; U.S. Pat. No. 7,041,870 describing K-M MOUSE (registered trademark) technology, and U.S. Patent Application Publication No. US 2007/0061900, describing VELOCIMOUSE (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucosedeficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

d) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In one embodiment, isolated nucleic acid encoding an anti-HLA-DQ2.5/8 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an anti-HLA-DQ2.5/8 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-HLA-DQ2.5/8 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

C. Assays

Anti-HLA-DQ2.5/8 antibodies provided herein may be identified, screened for, or characterized for their physical/chemical properties and/or biological activities by various assays known in the art.

1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with, for example, any of the above-mentioned DQN0016, DQN0089, DQN0092, DQN0100, DQN0157, DQN0139, and DQN 0177 antibodies for binding to HLA-DQ2.5/8. In certain embodiments, such a competing antibody binds to the same epitope (e.g., a linear or a conformational epitope) that is bound by the DQN0016, DQN0089, DQN0092, DQN0100, DQN0157, DQN0139, and DQN 0177 antibodies. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized HLA-DQ2.5/8 is incubated in a solution comprising a first labeled antibody that binds to HLA-DQ2.5/8 (e.g., the DQN0016, DQN0089, DQN0092, DQN0100, DQN0157, DQN0139, or DQN 0177 antibody) and a second unlabeled antibody that is being tested for its ability to compete with the first antibody for binding to HLA-DQ2.5/8. The second antibody may be present in a hybridoma supernatant. As a control, immobilized HLA-DQ2.5/8 is incubated in a solution comprising the first labeled antibody but not the second unlabeled antibody. After incubation under conditions permissive for binding of the first antibody to HLA-DQ2.5/8, excess unbound antibody is removed, and the amount of label associated with immobilized HLA-DQ2.5/8 is measured. If the amount of label associated with immobilized HLA-DQ2.5/8 is substantially reduced in the test sample relative to the control sample, then that indicates that the second antibody is competing with the first antibody for binding to HLA-DQ2.5/8. See Harlow and Lane (1988) Antibodies: A Laboratory Manual ch.14 (Cold Spring Harbor Laboratory, Cold Spring Harbor, N.Y.).

2. Activity Assays/Screening Method

In one aspect, assays are provided for identifying anti-HLA-DQ2.5/8 antibodies having a binding/biological activity. Such assays can be used in screening methods of the present invention.

In some embodiments, the present invention provides a method of screening for an anti-HLA-DQ2.5/8 antibody, which comprises: (a) testing whether an antibody has binding activity to HLA-DQ2.5 and HLA-DQ8; and selecting an antibody that has binding activity to HLA-DQ2.5 and HLA-DQ8; (b) testing whether an antibody has a specific binding activity to HLA-DR or DP; and selecting an antibody that has no specific binding activity to HLA-DR or DP; (c) testing whether an antibody has a specific binding activity to a complex of the invariant chain and HLA-DQ2.5/8; and selecting an antibody that has no specific binding activity to the complex of the invariant chain and HLA-DQ2.5/8. The method may further comprise testing whether an antibody has binding activity to monkey MHC-DQ; and selecting an antibody that has binding activity to monkey MHC-DQ.

In certain embodiments, the method of the present invention further comprises: testing whether an antibody binds to (or has binding activity to) HLA-DQ2.5/8 in the presence of a gluten peptide; and selecting an antibody that binds to (or has binding activity to) HLA-DQ2.5/8 in the presence of a gluten peptide. Preferably, the gliten peptide is gliadin.

In certain embodiments, the method of the present invention further comprises: testing whether an antibody has neutralizing activity against the binding between HLA-DQ2.5/8 and TCR; and selecting an antibody that has the neutralizing activity.

Before performing the steps below, candidate anti-HLA-DQ2.5/8 antibodies may be prepared by any methods, for example, as mentioned in Example 3.

Animals such as rabbits, mice, rats, and other animals suitable for immunization are immunized with an antigen (e.g., HLA-DQ2.5/8 optionally bound by a gliadin peptide). The antigen may be prepared as a recombinant protein using any methods, for example, as mentioned in Examples 1 and 2. Antibody-containing samples such as the blood and spleen are collected from the immunized animals. For B cell selection, for example, a biotinylated antigen is prepared, and antigen-binding B cells are bound by the biotinylated antigen, and the cells are subjected to cell sorting and culturing for selection. Specific binding of the cells to the antigen may be evaluated by any suitable method such as the ELISA method. This method may also be used for assessing the absence of cross-reactivity towards antigens of no interest. To isolate or determine the sequence of the selected antibody, for example, RNAs are purified from the cells, and DNAs encoding regions of the antibody are prepared by reverse transcription of the RNAs and PCR amplification. Furthermore, cloned antibody genes may be expressed in suitable cells, and the antibody may be purified from the culture supernatants for further analysis.

To test whether an anti-HLA-DQ2.5/8 antibody binds to an antigen of interest (e.g., HLA-DQ2.5, HLA-DQ8, monkey MHC-DQ, and HLA-DQ2.5/8 bound by a gluten peptide such as gliadin) or an antigen of no interest (e.g., HLA-DR, HLA-DP, the invariant chain, and a complex of the invariant chain and HLA-DQ2.5/8), any methods for assessing the binding can be used. For example, when an FACS-based cell sorting method is used, cells expressing the antigen (e.g., HLA-DQ2.5, HLA-DQ8, monkey MHC-DQ, HLA-DR, or HLA-DP) are incubated with the tested antibody, and then a suitable secondary antibody against the tested (i.e., primary) antibody is added and incubated. The binding between the antigen and the tested antibody is detected by FACS analysis using, for example, a chromogenic/fluorescent label attached to the secondary antibody (for example, as mentioned in Example 4.1). Alternatively, any of the measurement methods mentioned in “1. Antibody Affinity” in this specification can be utilized. For example, the measurement of Kd by a BIACORE surface plasmon resonance assay can be used for assessing the binding between the tested antibody and HLA-DQ in the presence of a gluten peptide (e.g., gliadin) or the invariant chain (for example, as mentioned in Examples 4.2 and 4.4).

In certain embodiments, the method of the present invention further comprises: testing whether the antibody has neutralizing activity against the binding between HLA-DQ2.5/8 and TCR (or the interaction between HLA-DQ2.5/8 and HLA-DQ2.5/8-restricted CD4+ T cells); and selecting the antibody that has the neutralizing activity. These steps can be performed in the presence of a gluten peptide such as a gliadin peptide, i.e., using HLA-DQ2.5/8 bound by the peptide. The neutralizing activity can be assessed, for example, as mentioned in Example 4.3. Briefly, beads such as streptavidin-coated yellow particles are appropriately prepared, and soluble HLA-DQ bound by a gliadin peptide is added to the beads for immobilization on a plate. The plate is washed and blocked, and the antibody is added thereto and incubated. When the binding between HLA-DQ2.5/8 and TCR is assessed, for example, D2 TCR tetramer-PE may be added and incubated. Binding between the two may be evaluated based on the chromogenic/fluorescent label of TCR bound by HLA-DQ2.5/8.

In certain embodiments, the method of the present invention further comprises: testing whether the antibody is internalized into the cell with the invariant chain; and selecting the antibody that is not (substantially) internalized into the cell with the invariant chain. The cell internalization (“rapid internalization” mentioned above) can be assessed by FACS analysis, for example, as mentioned in Example 4.5. Briely, a chromogenic/fluorescent label (e.g., AlexaFluor 555) is attached to the tested antibody, and this is incubated with suitable cells in the presence or absence of Cytochalasin D which blocks the delivery of class II/invariant chain complexes to lysosomes. Then, an appropriate secondary antibody against the tested antibody (i.e., primary antibody), for example, an anti-human IgG Fc antibody with FITC, is added and incubated. This is subjected to FACS measurement, and the rate of invariant chain-dependent cell internalization of the antibody is calculated from values obtained in the absence and presence of Cytochalasin D. If these values are equal or comparable to each other, it can be said that the antibody is not internalized with the invariant chain.

D. Therapeutic Methods and Compositions

Any of the anti-HLA-DQ2.5/8 antibodies provided herein may be used in therapeutic methods.

In one aspect, an anti-HLA-DQ2.5/8 antibody for use as a medicament is provided. In further aspects, an anti-HLA-DQ2.5/8 antibody for use in treating celiac disease is provided. In certain embodiments, an anti-HLA-DQ2.5/8 antibody for use in a method of treatment is provided. In certain embodiments, the invention provides an anti-HLA-DQ2.5/8 antibody for use in a method of treating an individual having celiac disease, which comprises administering to the individual an effective amount of the anti-HLA-DQ2.5/8 antibody. In one such embodiment, the method further comprises administering to the individual an effective amount of, for example, at least one additional therapeutic agent described below. In further embodiments, the invention provides an anti-HLA-DQ2.5/8 antibody for use in blocking the interaction between HLA-DQ2.5/8 and an HLA-DQ2.5/8-restricted CD4+ T cell and/or the interaction between HLA-DQ2.5/8 and a TCR. In certain embodiments, the invention provides an anti-HLA-DQ2.5/8 antibody for use in a method of blocking the interaction between HLA-DQ2.5/8 and an HLA-DQ2.5/8-restricted CD4+ T cell and/or the interaction between HLA-DQ2.5/8 and a TCR in an individual, which comprises administering to the individual an effective amount of the anti-HLA-DQ2.5/8 antibody to block the interaction between HLA-DQ2.5/8 and an HLA-DQ2.5/8-restricted CD4+ T cell and/or the interaction between HLA-DQ2.5/8 and a TCR. An “individual” according to any of the above embodiments is preferably a human.

In a further aspect, the invention provides use of an anti-HLA-DQ2.5/8 antibody in the manufacture or preparation of a medicament. In one embodiment, the medicament is for treatment of celiac disease. In a further embodiment, the medicament is for use in a method of treating celiac disease that comprises administering an effective amount of the medicament to an individual having celiac disease. In one such embodiment, the method further comprises administering to the individual an effective amount of, for example, at least one additional therapeutic agent described below. In a further embodiment, the medicament is for blocking the interaction between HLA-DQ2.5/8 and an HLA-DQ2.5/8-restricted CD4+ T cell and/or the interaction between HLA-DQ2.5/8 and a TCR. In a further embodiment, the medicament is for use in a method of blocking the interaction between HLA-DQ2.5/8 and an HLA-DQ2.5/8-restricted CD4+ T cell and/or the interaction between HLA-DQ2.5/8 and a TCR in an individual, which comprises administering to the individual an amount effective of the medicament to block the interaction between HLA-DQ2.5/8 and an HLA-DQ2.5/8-restricted CD4+ T cell and/or the interaction between HLA-DQ2.5/8 and a TCR. The “individual” according to any of the above embodiments may be a human.

In a further aspect, the invention provides a method for treating celiac disease. In one embodiment, the method comprises administering to an individual having celiac disease an effective amount of an anti-HLA-DQ2.5/8 antibody. In one such embodiment, the method further comprises administering to the individual an effective amount of at least one additional therapeutic agent described below. The “individual” according to any of the above embodiments may be a human.

In a further aspect, the invention provides a method for blocking the interaction between HLA-DQ2.5/8 and an HLA-DQ2.5/8-restricted CD4+ T cell and/or the interaction between HLA-DQ2.5/8 and a TCR in an individual. In one embodiment, the method comprises administering to the individual an effective amount of an anti-HLA-DQ2.5/8 antibody to block the interaction between HLA-DQ2.5/8 and an HLA-DQ2.5/8-restricted CD4+ T cell and/or the interaction between HLA-DQ2.5/8 and a TCR. In one embodiment, the “individual” is a human.

In a further aspect, the invention provides pharmaceutical formulations comprising any of the anti-HLA-DQ2.5/8 antibodies provided herein, e.g., for use in any of the above therapeutic methods. In one embodiment, a pharmaceutical formulation comprises any of the anti-HLA-DQ2.5/8 antibodies provided herein and a pharmaceutically acceptable carrier. In another embodiment, a pharmaceutical formulation comprises any of the anti-HLA-DQ2.5/8 antibodies provided herein and at least one additional therapeutic agent, e.g., as described below.

It is understood that any of the above formulations or therapeutic methods may be carried out using an immunoconjugate of the invention in place of or in addition to an anti-HLA-DQ2.5/8 antibody.

### EXAMPLES

The following are examples of methods and compositions of the invention. It is understood that various other embodiments may be practiced, given the general description provided above.

### Example 1

Expression and Purification of Recombinant Proteins

1.1. Expression and Purification of Recombinant HLA-DQ2.5/33Mer Gliadin Peptide

The sequences used for expression and purification are: HLA-DQA1*0501 (Protein Data Bank accession code 4OZG; IMGT/HLA accession No. HLA00613) and HLA-DQB1*0201 (Protein Data Bank accession code 4OZG; IMGT/HLA accession No. HLA00622), both of which have a CAMPATH-1H signal sequence: MGWSCIILFLVATATGVHS (SEQ ID NO: 67). HLA-DQA1*0501 has C47S mutation, GGGG linker (SEQ ID NO: 68) and c-fos leucine zipper sequence (PNAS, 1998 Sep. 29; 95(20): 11828-33) and a Flag-tag on the C-terminus of HLA-DQA1*0501. HLA-DQB1*0201 has 33 mer gliadin peptide sequence: LQLQPFPQPELPYPQPELPYPQPELPYPQPQPF (SEQ ID NO: 69), and factor X cleavage linker (Acta Crystallogr Sect F Struct Biol Cryst Commun. 2007 Dec. 1; 63(Pt 12): 1021-1025.) on the N-terminus of HLA-DQB1*0201, GGGGG linker (SEQ ID NO: 70) and c-jun leucine zipper sequence (PNAS, 1998 Sep. 29; 95(20): 11828-33), GGGGG linker, and BAP sequence (BMC Biotechnol. 2008; 8: 41), 8× His-tag on the C-terminus of HLA-DQB1*0201.

Recombinant HLA-DQ2.5/33 mer gliadin fusion peptide was expressed transiently using FreeStyle293-F cell line (Thermo Fisher). Conditioned media expressing HLA-DQ2.5/33 mer gliadin peptide was incubated with an immobilized metal affinity chromatography (IMAC) resin, followed by elution with imidazole. Fractions containing HLA-DQ2.5/33 mer gliadin peptide were collected and subsequently subjected to a Superdex 200 gel filtration column (GE healthcare) equilibrated with 1×PBS. Fractions containing HLA-DQ2.5/33 mer gliadin peptide were then pooled and stored at −80 degrees C. The purified HLA-DQ2.5/33 mer gliadin peptide was biotinylated using BirA (Avidity).

1.2. Expression and Purification of Recombinant HLA-DQ8/Gliadin Peptide

The sequences used for expression and purification are: HLA-DQA1*0301 (Protein Data Bank accession code 4GG6; IMGT/HLA accession No. HLA00608) and HLA-DQB1*0302 (Protein Data Bank accession code 4GG6; IMGT/HLA accession No. HLA00627), both of which have a CAMPATH-1H signal sequence: MGWSCIILFLVATATGVHS. HLA-DQA1*0301 has SSADLVPRGGGG linker (SEQ ID NO: 71) and c-fos leucine zipper sequence (PNAS, 1998 Sep. 29; 95(20): 11828-33) and a Flag-tag on the C-terminus of HLA-DQA1*0301. HLA-DQB1*0302 has gliadin peptide sequence: QQYPSGEGSFQPSQENPQ (SEQ ID NO: 72), and factor X cleavage linker (Acta Crystallogr Sect F Struct Biol Cryst Commun. 2007 Dec. 1; 63(Pt 12): 1021-1025.) on the N-terminus of HLA-DQB1*0302, SSADLVPRGGGGG linker (SEQ ID NO: 73) and c-jun leucine zipper sequence (PNAS, 1998 Sep. 29; 95(20): 11828-33), GGGGG linker, and BAP sequence (BMC Biotechnol. 2008; 8: 41), 8× His-tag on the C-terminus of HLA-DQB1*0201.

Recombinant HLA-DQ8/gliadin fusion peptide was expressed transiently using FreeStyle293-F cell line. Conditioned media expressing HLA-DQ8/gliadin peptide was incubated with an IMAC resin, followed by elution with imidazole. Fractions containing HLA-DQ8/gliadin peptide were collected and subsequently subjected to a Superdex 200 gel filtration column equilibrated with 1×PBS. Fractions containing HLA-DQ8/gliadin peptide were then pooled and stored at −80 degrees C.

1.3. Expression and Purification of Recombinant HLA-DQ2.5/Invariant Chain Complex

The sequences used for expression and purification are: HLA-DQA1*0501 (Protein Data Bank accession code.4OZG; IMGT/HLA accession No. HLA00613), HLA-DQB1*0201 (Protein Data Bank accession code 4OZG; IMGT/HLA accession No. HLA00622), the invariant chain (76-295) (GenBank accession No. NM_001025159/NP_001020330 for the full-length invariant chain), all of which have a CAMPATH-1H signal sequence: MGWSCIILFLVATATGVHS. HLA-DQA1*0501 has C47S mutation, GGGG linker and c-fos leucine zipper sequence (PNAS, 1998 Sep. 29; 95(20): 11828-33) on the C-terminus of HLA-DQA1*0501. HLA-DQB1*0201 has GGGGG linker and c-jun leucine zipper sequence (PNAS, 1998 Sep. 29; 95(20): 11828-33), and 8× His-tag on the C-terminus of HLA-DQB1*0201. The invariant chain (76-295) has a Flag-tag, GCN4 variant amino acid sequence (Science. 1993 Nov. 26; 262(5138):1401-7), and GGGGS linker (SEQ ID NO: 74) on the N-terminus of the invariant chain (76-295).

Recombinant recombinant HLA-DQ2.5/invariant chain complex was expressed transiently using FreeStyle293-F cell line. Conditioned media expressing recombinant HLA-DQ2.5/invariant chain complex was incubated with an IMAC resin, followed by elution with imidazole. Fractions containing recombinant HLA-DQ2.5/invariant chain complex were collected and subsequently subjected to a Superose 6 gel filtration column (GE healthcare) equilibrated with 1×PBS. Fractions containing recombinant HLA-DQ2.5/invariant chain complex were then pooled and stored at −80 degrees C.

1.4. Expression and Purification of Recombinant TCRs

The sequences used for expression and purification are: S2 TCR alpha chain (Protein Data Bank accession code 40ZI), S2 TCR beta chain (Protein Data Bank accession code 40ZI), D2 TCR alpha chain (Protein Data Bank accession code 4OZG), D2 TCR beta chain (Protein Data Bank accession code 4OZG), SP3.4 TCR alpha chain (Protein Data Bank accession code 4GG6), SP3.4 TCR beta chain (Protein Data Bank accession code 4GG6). S2 TCR alpha chain has a CAMPATH-1H signal sequence: MGWSCIILFLVATATGVHS, and BAP sequence (BMC Biotechnol. 2008; 8: 41), 8× His-tag on the C-terminus of S2 TCR alpha chain. S2 TCR beta chain has a CAMPATH-1H signal sequence: MGWSCIILFLVATATGVH (SEQ ID NO: 75), and Flag-tag on the C-terminus of S2 TCR beta chain. D2 TCR alpha chain has a signal sequence derived from rat serum albumin: MKWVTFLLLLFISGSAFS (SEQ ID NO: 76), and BAP sequence (BMC Biotechnol. 2008; 8: 41), 8× His-tag on the C-terminus of D2 TCR alpha chain. D2 TCR beta chain has a signal sequence derived from rat serum albumin: MKWVTFLLLLFISGSAFS, and Flag-tag on the C-terminus of D2 TCR beta chain. SP3.4 TCR alpha chain has a CAMPATH-1H signal sequence: MGWSCIILFLVATATGVHS, and BAP sequence (BMC Biotechnol. 2008; 8: 41), 8× His-tag on the C-terminus of SP3.4 TCR alpha chain. SP3.4 TCR beta chain has a CAMPATH-1H signal sequence: MGWSCIILFLVATATGVHS, and Flag-tag on the C-terminus of SP3.4 TCR beta chain.

Recombinant soluble TCR protein was expressed transiently using FreeStyle293-F cell line. Conditioned media expressing TCR protein was applied to a column packed with anti-Flag M2 affinity resin (Sigma) and eluted with Flag peptide (Sigma). Fractions containing TCR protein were collected and subsequently applied to a column packed with an IMAC resin, followed by elution with imidazole. Fractions containing TCR protein were collected and subsequently subjected to a Superdex 200 gel filtration column equilibrated with 1×PBS. Fractions containing TCR protein were then pooled and stored at −80 degrees C.

The purified TCR protein was biotinylated using BirA, then combined with PE-labeled streptavidin (BioLegend) to form tetrameric TCR protein.

### Example 2

Establishment of Ba/F3 Cell Lines Expressing HLA-DQ2.5, HLA-DQ8, HLA-DR, HLA-DP, and cynomolgus monkey MHC-DQ allele #1, #2, and #3

HLA-DQA1*0501 cDNA (IMGT/HLA accession No. HLA00613), HLA-DQA1*0301 cDNA (IMGT/HLA accession No. HLA00608), HLA-DRA1*0101 cDNA (GenBank accession No. NM_019111.4), or HLA-DPA1*0103 cDNA (IMGT/HLA accession No. HLA00499) was inserted into the expression vector pCXND3 (WO/2008/156083).

HLA-DQB1*0201 cDNA (IMGT/HLA accession No. HLA00622), HLA-DQB1*0302 cDNA (IMGT/HLA accession No. HLA00627), HLA-DRB1*0301 cDNA (IMGT/HLA accession No. HLA00671), or HLA-DPB1*0401 cDNA (IMGT/HLA accession No.HLA00521) was inserted into the expression vector pCXZD1 (US/20090324589).

Three different cynomolgus MHC-DQA (SEQ ID NO: 77 for allele #1; and SEQ ID NO: 79 for alleles #2 and #3) and DQB (SEQ ID NO: 78 for allele #1; SEQ ID NO: 80 for allele #2; and SEQ ID NO: 81 for allele #3) sequences was read by PCR from cynomolgus monkey PBMC, and the expression vectors were constructed in the same manner.

200 ng each of the linearized HLA-DQA1*0501-pCXND3 and HLA-DQB1*0201-pCXZD1, HLA-DQA1*0301-pCXND3 and HLA-DQB1*0302-pCXZD1, HLA-DRA1*0101-pCXND3 and HLA-DRB1*0301-pCXZD1, HLA-DPA1*0103-pCXND3 and HLA-DPB1*0401-pCXZD1, cynomolgus monkey MHC DQA1*M2-1-pCXND3 and cynomolgus monkey MHC DQB1*M2-1-pCXZD1, cynomolgus monkey MHC DQA1*M3-1-pCXND3 and cynomolgus monkey MHC DQB1*M3-1-pCXZD1, cynomolgus monkey MHC DQA1*M3-1-pCXND3 and cynomolgus monkey MHC DQB1*M3-2-pCXZD1 were simultaneously introduced into mouse IL-3-dependent pro-B cell-derived cell line Ba/F3 by electroporation (LONZA, 4D-Nucleofector X).

After introduction, Geneticin and Zeocin were added, and the cells were cultured to obtain a cell line that resistant to Geneticin and Zeocin. Transfected cell lines were plated in a 96-well by single-cell sorting with AriaIII (Becton Dickinson) and expanded. Established each cell lines were named Ba/F3-HLA-DQ2.5 (HLA-DQA1*0501, HLA-DQB1*0201), Ba/F3-HLA-DQ8 (HLA-DQA1*0301, HLA-DQB1*0302), Ba/F3-HLA-DR (HLA-DRA1*0101, HLA-DRB1*0301), Ba/F3-HLA-DP (HLA-DPA1*0103, HLA-DPB1*0401), Ba/F3-cynoDQ C1 (cynomolgus monkey MHC DQA1*M2-1, cynomolgus monkey MHC DQB1*M2-1), Ba/F3-cynoDQ C2 (cynomolgus monkey MHC DQA1*M3-1, cynomolgus monkey MHC DQB1*M3-1), Ba/F3-cynoDQ C3 (cynomolgus monkey MHC DQA1*M3-1, cynomolgus monkey MHC DQB1*M3-2).

### Example 3

Generation of Anti-DQ2.5 Antibodies

Anti-DQ2.5 antibodies were prepared, selected and assayed as follows:

NZW rabbits were immunized intradermally with HLA-DQ2.5/33 mer gliadin peptide. Four repeated doses were given over a 2-month period followed by blood and spleen collection. For B-cell selection, biotinylated HLA-DQ2.5/33 mer gliadin peptide was prepared. Antigen binding B-cells were stained with the biotinylated antigen, sorted using a cell sorter and then plated and cultured according to the procedure described in WO2016098356A1. After cultivation, the B-cell culture supernatants were collected for further analysis and the B-cell pellets were cryopreserved.

Specific binding to antigen was evaluated by ELISA using the B cell culture supernatants. The results showed that 9,841 B cell lines exhibited binding to HLA-DQ2.5/33 mer gliadin peptide.

We have also characterized binding to HLA-DQ8 by cell-based ELISA according to the procedure described in BioTechniques 2003, 35:1014-1021 using the selected B cell supernatant and HLA-DQ8 expressing Ba/F3 cell line (Ba/F3-HLA-DQ8) described above. The results showed that 701 B cell lines could bind to HLA-DQ8.

In order to evaluate the cross-reactivity to HLA-DR, we have conducted cell-based ELISA using the selected 701 B cell supernatants and HLA-DR expressing Ba/F3 cell line (Ba/F3-HLA-DR) described above.

Finally cross-reactivity to cynoDQ C1, cynoDQ C2 and cynoDQ C3 were checked by flow cytometry using selected B cell supernatant with Ba/F3-cynoDQ C1, Ba/F3-cynoDQ C2 and Ba/F3-cynoDQ C3 cell lines prepared above. B cells without binding to HLA-DR but with good cross-reactivity to cynoDQ were preferred and selected for cloning.

The RNAs of 188 B cell lines with desired binding specificities were purified from the cryopreserved cell pellets using the ZR-96 Quick-RNA kits (ZYMO RESEARCH, Cat No. R1053). These were named as DQN0001-0188. DNAs encoding antibody heavy-chain variable regions in the selected lines were amplified by reverse transcription PCR and recombined with a DNA encoding the F1332m heavy-chain constant region (SEQ ID NO: 65). DNAs encoding antibody light-chain variable regions were also amplified by reverse transcription PCR and recombined with a DNA encoding the hkOMC light-chain constant region (SEQ ID NO: 66). Cloned antibodies were expressed in Freestyle™ 293-F Cells (Invitrogen) and purified from culture supernatants. Through further evaluation described below, five clones (DQN0016, DQN0092, DQN0100, DQN0139 and DQN0157) are selected based on binding ability, specificity and functionality. Two clones (DQN0089 and DQN0177) were used as assay controls. The VH and VL sequences of these seven antibodies are shown in Table 1. The sequence ID numbers of VH, VL, HCDRs and LCDRs of these seven antibodies are listed in Table 2. The sequences of these CDRs are shown in Table 1. Elsewhere in the specification and drawings, DQN0016, DQN0089, DQN0092, DQN0100, DQN0139, DQN0157 and DQN0177 may be alternatively called DQN0016cc, DQN0089ff, DQN0092hh, DQN0100aa, DQN0139bb, DQN0157cc and DQN0177aa, respectively.

TABLE 1

| | FR1 | CDR1 | FR2 |
|---|---|---|---|
| Name | 0 1 2 3 | 3 | 3 4 |
| (VH) | 1 2 3 4 5 6 7 8 9 0 1 2 3 4 5 6 7 8 9 0 1 2 3 4 5 6 7 8 9 0 | 1 2 3 4 5 a b | 6 7 8 9 0 1 2 3 4 5 6 7 8 9 |
| DQN0016 | Q - S L E E S G G D L V K P G A S L T L T C T A S G F S F S | S R Y W M C - | W V R Q A P G K G L E W I A |
| DQN0089 | Q E Q V V E Y G G D L V Q P E G S L T L T C K A S G L D F S | S T Y Y M C - | W V R Q A P G K G L E W I A |
| DQN0092 | Q - S V E E S G G R L V T P G T P L T L T C T V S G F S L S | S Y Y I S - - | W V R Q A P G K G L E W I G |
| DQN0100 | Q Q Q L E E S G G G L V K P G A S L T L T C K G S G F S F T | S G Y W I C - | W V R Q A P G K G L E W I G |
| DQN0157 | Q - S L E E S G G D L V K P G A S L T L T C T A S G F S F S | S S Y Y M C - | W V R Q A P G K G L E W I A |
| DQN0139 | Q - S L E E S G G R L V T P G T A L T L T C T V S G F S L S | S Y A M G - - | W V R Q A P G K G L E Y I G |
| DQN0177 | Q - S V E E S G G R L V T P G T P L T L T C T V S G F S L S | S Y A M S - - | W V R Q A P G K G L E W I G |

| | CDR2 | CDR3 | FR4 |
|---|---|---|---|
| Name | 5 6 | 9 10 | 1 1 |
| (VH) | 0 1 2 a b c 3 4 5 6 7 8 9 0 1 2 3 4 5 | 5 6 7 8 9 0 a b c d e f g h i j k l 1 2 | 3 4 5 6 7 8 9 0 1 2 3 |
| DQN0016 | C I D A G - S S G I T Y Y A S W A N G | D A W S T T D G W N T F - - - - - - N L | W G P G T L V T V S S |
| DQN0089 | C I Y G G - S S D S T Y Y A S W A K G | Y D Y G A V G Y - - - - - - - - - - D L | W G P G T L V T V S S |
| DQN0092 | I I Y - - - T D G V T D Y P N W A K G | D A A S V Y G C G Y F - - - - - - - D L | W G P G T L V T V S S |
| DQN0100 | C I Y T G - S S G S T Y Y A S W A K G | D P SG A S S G W D - - - - - - - - N L | W G P G T L V T V S S |
| DQN0157 | C I Y T G - S S G S T Y Y P S W A K G | V R A G S G Y Y Y G - - - - - - - - N L | W G P G T L V T V S S |
| DQN0139 | W T S - - - P G D S A Y Y A S W T K G | D A A Y I G H W A F - - - - - - - - N L | W G P G T L V T V S S |
| DQN0177 | V I S - - - Y S G R T Y D A S W A K G | V G D S Y G Y A Y A T V I F T Y H F N L | W G P G T L V T V S S |

| | FR1 | CDR1 | FR2 |
|---|---|---|---|
| Name | 0 1 2 | 2 3 | 3 4 |
| (VH) | 1 2 3 4 5 6 7 8 9 0 1 2 3 4 5 6 7 8 9 0 1 2 3 | 4 5 6 7 a b c d e f 8 9 0 1 2 3 4 | 5 6 7 8 9 0 1 2 3 4 5 6 7 8 9 |
| DQN0016 | D V V M T Q T P A S V S A A V G G T V T I K C | Q A S Q - - - - - - S I G S N L N | W Y Q Q K P G Q S P K L L I Y |
| DQN0089 | E V V V T Q T P A S V E V A V G G T V T I K C | Q A S Q - - - - - - N I S P Y L S | W Y Q Q K P G Q P P K L L I Y |
| DQN0092 | T F K L T Q T P A S V E A A V G G T V T I K C | Q A S E - - - - - - S N G N A L A | W Y Q Q K P G Q P P K L L I Y |
| DQN0100 | A F E L T Q T P S S V E A A V G G T V T I K C | Q A S Q - - - - - - T I V S W L A | W Y Q Q K P G Q R P K L L I L |
| DQN0157 | D I V M T Q T P S S V S A A V G G T V T I K C | Q A S Q - - - - - - T I Y S G L A | W Y Q Q K P G Q P P K L L I Y |
| DQN0139 | D V V M T Q T P A S V S A P V G G T V T I N C | Q A S E - - - - - - S I Y S N L A | W Y Q Q K P G Q P P K L L I Y |
| DQN0177 | D P V L T Q T P S S A S E P V G G T V T I K C | Q A S E - - - - - - S I S S S L A | W Y Q Q K P G Q R P K L L I Y |

| | CDR2 | FR3 | CDR3 | FR4 |
|---|---|---|---|---|
| Name | 5 | 5 6 7 8 | 9 | 1 0 |
| (VL) | 0 1 2 3 4 5 6 | 7 8 9 0 1 2 3 4 5 6 7 8 9 0 1 2 3 4 5 6 7 8 9 0 1 2 3 4 5 6 7 8 | 9 0 1 2 3 4 5 a b c d e f 6 7 | 8 9 0 1 2 3 4 5 6 7 |
| DQN0016 | S A S T L A S | G V P S R F K G S G S G T E Y T L T I S N L E C A D A A T Y Y C | Q S Y Y Y T G S S A V - - N N | F G G G T K V E I K |
| DQN0089 | K A S T L A S | G V S S R F K G S G S G T Q F T L T I S D L E C A D A A T Y Y C | Q N N Y G V S I N Y G - - H T | F G G G T K V E I K |
| DQN0092 | R A F T L A S | G V P S R L K G S G S G T Q F A L T I S D L E C A D A A T Y Y C | Q T Y Y G S N T D F - - - N A | F G G G T K V E I K |
| DQN0100 | Y A S N L A S | G V S S R F R G S G S G T D Y T L T I S D L E C A D A A T Y Y C | Q S Y Y F S T S S P D M - Y A | F G G G T K V E I K |
| DQN0157 | D A S D L A S | G V P S R F S G S G S G T E F T L A I S G V Q C A D A A T Y Y C | H G D Y Y G G S G T F F - W T | F G G G T K V E I K |
| DQN0139 | G A S T L E S | G V P S R V K G S G Y G T Q F T L T I S D L E C A D A A T Y Y C | Q Q Y Y G S S S T A - - - F T | F G G G T K V E I K |
| DQN0177 | D A S K L A S | G V P S R F K G S G S G T E F T L T I S G V Q C A D A A T Y Y C | Q H G L S S G S T N R - - C A | F G G G T K V E I K |

TABLE 2

| Anti- | | | | | | | | |
|---|---|---|---|---|---|---|---|---|
| body | SEQ ID NO: | | | | | | | |
| Name | VH | HCDR1 | HCDR2 | HCDR3 | VL | LCDR1 | LCDR2 | LCDR3 |
| DQN | 1 | 9 | 17 | 25 | 33 | 41 | 49 | 57 |
| 0016 | | | | | | | | |
| DQN | 2 | 10 | 18 | 26 | 34 | 42 | 50 | 58 |
| 0089 | | | | | | | | |
| DQN | 3 | 11 | 19 | 27 | 35 | 43 | 51 | 59 |
| 0092 | | | | | | | | |
| DQN | 4 | 12 | 20 | 28 | 36 | 44 | 52 | 60 |
| 0100 | | | | | | | | |
| DQN | 5 | 13 | 21 | 29 | 37 | 45 | 53 | 61 |
| 0157 | | | | | | | | |
| DQN | 6 | 14 | 22 | 30 | 38 | 46 | 54 | 62 |
| 0139 | | | | | | | | |
| DQN | 8 | 16 | 24 | 32 | 40 | 48 | 56 | 64 |
| 0177 | | | | | | | | |

### Example 4

Characterization of Anti-HLA-DQ2.5/8 Antibodies

4.1. Binding Analysis of the Antibodies to HLA-DQ2.5, HLA-DQ8, HLA-DR, and HLA-DP

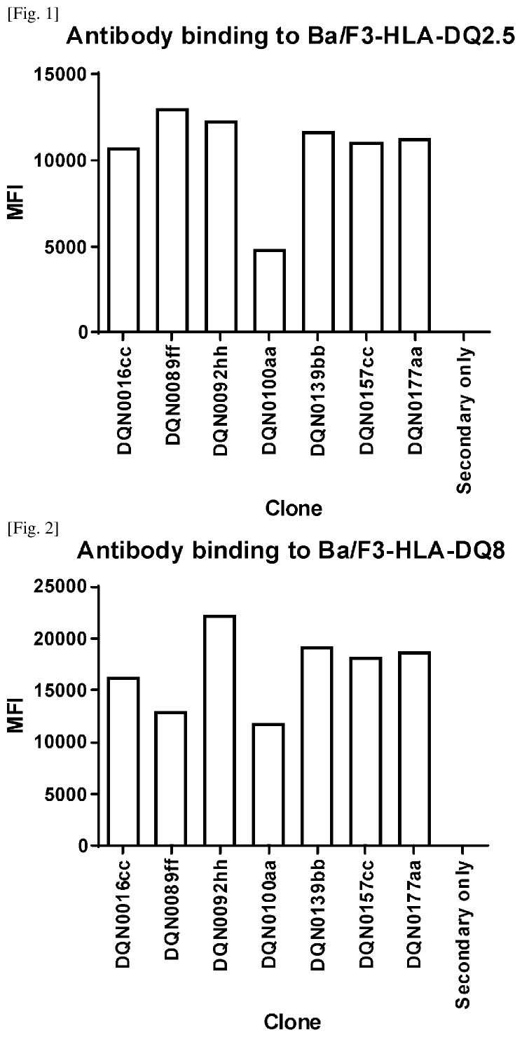

FIGS. 1 to 5 show binding of the anti-HLA-DQ2.5/8 antibodies to multiple MHC class II-expressing Ba/F3 cell lines as determined by FACS. Binding of antiHLA-DQ2.5/8 antibodies to HLA-DQ2.5-expressing Ba/F3 cells (Ba/F3-HLA-DQ2.5), Ba/F3-HLA-DQ8, Ba/F3-HLA-DR, Ba/F3-HLA-DP, Ba/F3-cynoDQ C1, Ba/F3-cynoDQ C2, and Ba/F3-cynoDQ C3 was tested.

Anti-HLA-DQ2.5/8 antibodies were incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C. and washed with FACS buffer. Data acquisition was performed on an FACS Verse (Becton Dickinson), followed by analysis using the FlowJo software (Tree Star) and the GraphPad Prism software (GraphPad).

FIGS. 1 and 2 show that all of the anti-HLA-DQ2.5/8 antibodies produced in Example 3, i.e., DQN0016, DQN0089, DQN0092, DQN0100, DQN0139, DQN0157 and DQN0177, bind to HLA-DQ2.5 and HLA-DQ8 which are the antigens of interest. In particular, DQN0016, DQN0092, DQN0139, DQN0157 and DQN0177 strongly bind to these HLA molecules. In the figures, “Secondary only” means that the antiHLA-DQ2.5/8 antibody was not added in the experiments, and this column shows the negative control (background) level. The same applies to FIGS. 3 to 5.

Meanwhile, FIGS. 3 and 4 show that only DQN0089 also binds to HLA-DR and HLA-DP. These results indicate that the rest of the tested antibodies, i.e., DQN0016, DQN0092, DQN0100, DQN0139, DQN0157 and DQN0177, specifically bind to HLA-DQ2.5/8.

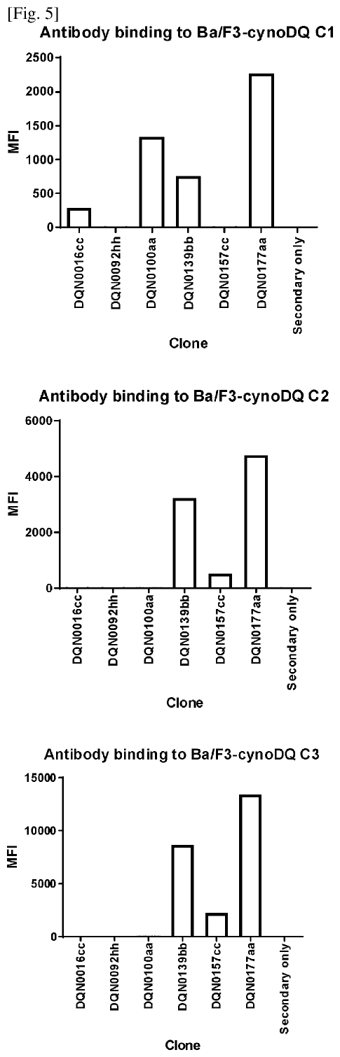

FIG. 5 indicates that DQN0092 does not significantly bind to any of the tested cynomolgus monkey MHC-DQ molecules, but DQN0016, DQN0089, DQN0100, DQN0139, DQN0157 and DQN0177 bind to at least one of the cynomolgus MHC-DQ molecules. These results show that DQN0016, DQN0089, DQN0100, DQN0139, DQN0157 and DQN0177 are more preferable in that they bind to the cynomolgus MHC-DQ molecules which are highly homologous to the human counterparts, thus are expected to have a stronger binding activity towards the human HLA molecules.

4.2. Binding Analysis of the Antibodies to HLA-DQ2.5/Gliadin Peptide and HLA-DQ8/Gliadin Peptide

The affinity of the anti-HLA-DQ2.5/8 antibodies for binding to human HLA-DQ2.5/gliadin peptide and HLA-DQ8/gliadin peptide at pH 7.4 was determined at 37 degrees C. using the Biacore T200 instrument (GE Healthcare). Anti-human Fc (GE Healthcare) was immobilized onto all flow cells of a CM4 sensor chip using an amine coupling kit (GE Healthcare). All antibodies and analytes were prepared in ACES, pH 7.4 containing 20 mM ACES, 150 mM NaCl, 0.05% Tween 20, 0.005% NaN₃. Each antibody was captured onto the sensor surface by anti-human Fc. Antibody capture levels were aimed at 200 resonance units (RUs). A recombinant human HLA-DQ2.5/gliadin peptide was injected at 100 nm and 400 nM prepared by four-fold serial dilution, followed by dissociation, whereas a recombinant human HLA-DQ8/gliadin peptide was injected at 50 to 200 nM prepared by four-fold serial dilution, followed by dissociation. The sensor surface was regenerated each cycle with 3M MgCl₂. Binding affinity was determined by processing and fitting the data to a 1:1 binding model using the Biacore T200 Evaluation software, version 2.0 (GE Healthcare). A commercially available antibody (SPV-L3) was tested as comparison.

The affinity of the anti-HLA-DQ2.5/8 antibodies for binding to HLA-DQ2.5/gliadin peptide or HLA-DQ8/gliadin peptide is shown in Table 3. The results indicate that all of the tested antibodies (i.e., DQN0016, DQN0092, DQN0100, DQN0157, DQN0139 and DQN0177) bind to the HLA-DQ2.5/gliadin peptide molecule and the HLA-DQ8/gliadin peptide molecule. All of the antibodies have a much stronger affinity than SPVL3 for binding to HLA-DQ2.5/gliadin peptide. Additionally, these antibodies have a stronger affinity than SPV-L3 for binding to HLA-DQ8/gliadin peptide, except for DQN0016.

These results demonstrate that the anti-HLA-DQ2.5/8 antibodies of the present invention bind to the HLA molecules in the presence of gliadin, i.e., bind to the HLA molecules bound by gliadin.

TABLE 3

| | Human HLADQ2.5 gliadin | | | Human HLADQ8 | | |
|---|---|---|---|---|---|---|
| Ab name | ka (M⁻¹s⁻¹) | kd (s⁻¹) | KD (M) | ka (M⁻¹s⁻¹) | kd (s⁻¹) | KD (M) |
| DQN0016cc | 9.12E + 04 | 3.31E − 04 | 3.62E − 09 | 2.63E + 05 | 3.96E − 02 | 1.51E − 07 |
| DQN0092hh | 7.63E + 04 | 1.47E − 04 | 1.93E − 09 | 1.68E + 05 | 1.22E − 03 | 7.28E − 09 |
| DQN0100aa | 1.44E + 05 | 6.76E − 04 | 4.69E − 09 | 2.36E + 05 | 4.58E − 04 | 1.94E − 09 |
| DQN0157cc | 5.84E + 04 | 3.63E − 04 | 6.21E − 09 | 1.46E + 05 | 3.30E − 03 | 2.26E − 08 |
| DQN0139bb | 6.88E + 04 | 1.89E − 04 | 2.74E − 09 | 7.94E + 04 | 9.01E − 04 | 1.13E − 08 |
| DQN0177aa | 4.48E + 05 | 7.44E − 04 | 1.66E − 09 | 3.74E + 05 | 1.37E − 03 | 3.67E − 09 |
| SPV-L3 | 2.47E + 04 | 2.32E − 03 | 9.38E − 08 | 7.84E + 04 | 5.03E − 03 | 6.42E − 08* |

*The data for SPV-L3 was measured using protein A/G capture method

4.3. Neutralizing Assay of the Antibodies

FIG. 6 shows neutralizing activity of the anti-HLA-DQ2.5/8 antibodies. Streptavidin-coated yellow particles (Spherotech, SVFB-2552-6K) were incubated in blocking buffer (2% BSA in PBS) for 30 minutes with shaking at room temperature. After centrifugation and aspiration of the supernatant, 0.167 micro g/mL of soluble HLA-DQ2.5/33 mer gliadin peptide was then added as 1.2E+4 beads/micro L solution and immobilized for 60 minutes with shaking at room temperature in 96 well plates (Sigma Aldrich, Cat No. M2686). The final concentration of HLA-DQ2.5/33 mer gliadin peptide was 0.075 micro g/mL for the D2 TCR neutralizing assay and 0.375 micro g/mL for the S2 TCR neutralizing assay. The final concentration of HLA-DQ8/gliadin peptide was 0.075 micro g/mL. The plates were washed with blocking buffer, and serially diluted anti-HLA-DQ2.5/8 antibodies were added and incubated for 60 minutes with shaking at room temperature. D2 TCR, S2 TCR tetramer-PE were then added to HLA-DQ2.5/33 mer gliadin peptide coated beads; and SP3.4 TCR was then added to HLA-DQ8/gliadin peptide-coated beads and incubated for 60 minutes with shaking at 4 degrees C., and washed with blocking buffer. The final concentration was 0.15 micro g/mL for D2 TCR tetramer-PE, 2.0 micro g/mL for S2 TCR tetramer-PE, and 0.15 micro g/mL for SP3.4 TCR-PE. Data acquisition was performed on LSR Fortessa (Becton Dickinson), followed by analysis using the FlowJo software (Tree Star) and the GraphPad Prism software (GraphPad).

As a result, DQN0016, DQN0100, DQN0139, DQN0157, and DQN0177 showed neutralizing activities against the binding between gliadin-bound HLA-DQ2.5 and D2 TCR, and also the binding between gliadin-bound HLA-DQ2.5 and S2 TCR. The activities of DQN0016, DQN0100, DQN0139, and DQN0157 were particularly strong. Additionally, DQN0016, DQN0100, DQN0139, DQN0157, and DQN0177 showed neutralizing activities against the binding between gliadin-bound HLA-DQ8 and SP3.4 TCR. The activities of DQN0016, DQN0100, DQN0139, and DQN0157 were particularly strong.

Thus, it was indicated that the antibodies of the present invention can block the interaction between HLA-DQ2.5/8 and an HLA-DQ2.5/8-restricted CD4+ T cell.

4.4. Binding Analysis of the Antibodies to HLA-DQ2.5/Invariant Chain

The binding response of the anti-HLADQ2.5 antibodies to human HLA-DQ/invariant chain at pH 7.4 was determined at 25 degrees C. using the Biacore T200 instrument (GE Healthcare). Anti-human Fc (GE Healthcare) was immobilized onto all flow cells of a CM4 sensor chip using an amine coupling kit (GE Healthcare). All antibodies and analytes were prepared in ACES, pH 7.4 containing 20 mM ACES, 150 mM NaCl, 0.05% Tween 20, 0.005% NaN₃. Each antibody was captured onto the sensor surface by anti-human Fc. Antibody capture levels were aimed at 200 resonance units (RUs). A recombinant human HLA-DQ/invariant chain (fusion protein) was injected at 100 nM, followed by dissociation. The sensor surface was regenerated each cycle with 3M MgCl₂.

The binding level of the anti-HLA-DQ2.5 antibodies towards human HLA-DQ2.5/invariant chain was monitored from the binding response. The binding level was normalized to the capture level of the corresponding anti-HLA-DQ2.5 antibody.

Since the amount of the antibody captured on the tip varied, the ratio of the binding level to the capture level was used for evaluation of the binding activity. As shown in FIG. 7, no significant binding towards HLA-DQ2.5/invariant chain was observed for DQN0016, DQN0092, DQN0100, DQN0139, and DQN0157. Thus, it is thought that the antibodies of the present invention do not specifically bind to the complex of the invariant chain and HLA-DQ.

As mentioned above, when HLA-DQ forms a complex with the invariant chain, the complex on the cell surface is rapidly internalized into the endosome (“rapid internalization”, which T1/2 is around 3.2 min). After degradation of the invariant chain in the endosome, the HLA-DQ/peptide complex is transferred to the cell surface, and then recognized by TCR on T cells. The complex without the invariant chain is slowly internalized into the endosome (“slow internalization”, where T_{1/2} is 789-1500 min).

The fact that the antibodies of the present invention do not significantly bind to HLA-DQ in the presence of the invariant chain indicates that the antibodies are less susceptible to rapid internalization which can cause the antibodies to be quickly transferred to the endosome and degraded. The absence of rapid cell internalization (i.e., rapid endosomal degradation) of the antiboies of the present invention is thought to be useful for treating celiac disease.

4.5. Cell Based Internalization Assay

The rate of the HLA-DQ/invariant chain complex-mediated internalization of the anti-HLA-DQ antibodies was determined.

The anti-HLA DQ antibodies were labeled with AlexaFluor 555 (Alexa Fluor (registered trademark) 555 Antibody Labeling Kit, Molecular Probes, Cat. A20187). Raji cells were pre-cultured with or without 10 micro M of Cytochalasin D from Zygosporium mansonii (SIGMA, Cat. C8273) and 100 micro M of Dynasore (SIGMA, Cat. D7693) for 1 hour at 37 degrees C. AlexaFluor 555-labeled anti-HLA-DQ antibodies were then added at a concentration of 2.5 micro g/mL and incubated with Raji cells for 2 days at 37 degrees C. After washing with FACS buffer (2% FBS, 2 mM EDTA in PBS), an FITC-labeled anti-human IgG Fc antibody was added and incubated for 20 minutes at 4 degrees C. and washed with FACS buffer. Data acquisition was performed on LSR Fortessa (Becton Dickinson), followed by analysis using the FlowJo software (Tree Star) and the GraphPad Prism software (GraphPad). The rate of the HLA-DQ/invariant chain complex-mediated internalization of the antiHLA-DQ antibodies was calculated by dividing the MFI value obtained in the absence of Cytochalasin D by the MFI value obtained in the presence of Cytochalasin D ([MFI (PE/FITC) without Cytochalasin D, Dynasore]/[MFI (PE/FITC) with Cytochalasin D, Dynasore]).

Cytochalasin D inhibits the invariant chain-mediated rapid internalization. Thus, if the above ratio is relatively low, it can be said that the antibody is not internalized with the invariant chain. As shown in FIG. 8, the ratios of DQN0016, DQN0089, DQN0092, DQN0100, DQN0138, and DQN0157 were aroud 3, especially the ratios of DQN0016, DQN0092, DQN0100, DQN0138, and DQN0157 were less than 2.5. Therefore, it is thought that the antibodies of the present invention do not specifically bind to a complex of the invariant chain and HLA-DQ, and do not undergo rapid internalization mediated by the invariant chain.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An anti-HLA-DQ2.5/8 antibody, wherein
a) the antibody has binding activity to HLA-DQ2.5 and HLA-DQ8,
b) the antibody has substantially no binding activity to HLA-DR or HLA-DP, and
c) the antibody has substantially no binding activity to a complex of invariant chain and HLA-DQ2.5.

2. The antibody of claim 1, wherein the antibody has binding activity to HLA-DQ2.5 and HLA-DQ8 in the presence of a gluten peptide.

3. The antibody of claim 2, wherein the gluten peptide is gliadin.

4. The antibody of claim 1, wherein the antibody has binding activity to monkey MHC-DQ.

5. The antibody of claim 1, wherein the antibody blocks the interaction between HLA-DQ2.5 and an HLA-DQ2.5 restricted CD4+ T cell, and the antibody blocks the interaction between HLA-DQ8 and an HLA-DQ8 restricted CD4+ T cell.

6. An anti-HLA-DQ2.5/8 antibody, wherein the antibody binds to HLA-DQ2.5 and HLA-DQ8 in the presence of a gluten peptide without cell internalization mediated by an invariant chain.

7. The antibody of claim 1, which is any one of (1) to (16) below:
(1) an antibody comprising the HCDR1 sequence of SEQ ID NO: 9, the HCDR2 sequence of SEQ ID NO: 17, the HCDR3 sequence of SEQ ID NO: 25, the LCDR1 sequence of SEQ ID NO: 41, the LCDR2 sequence of SEQ ID NO: 49, and the LCDR3 sequence of SEQ ID NO: 57;
(2) an antibody comprising the VH sequence of SEQ ID NO: 1 and the VL sequence of SEQ ID NO: 33;
(3) an antibody comprising the HCDR1 sequence of SEQ ID NO: 10, the HCDR2 sequence of SEQ ID NO: 18, the HCDR3 sequence of SEQ ID NO: 26, the LCDR1 sequence of SEQ ID NO: 42, the LCDR2 sequence of SEQ ID NO: 50, and the LCDR3 sequence of SEQ ID NO: 58;
(4) an antibody comprising the VH sequence of SEQ ID NO: 2 and the VL sequence of SEQ ID NO: 34;
(5) an antibody comprising the HCDR1 sequence of SEQ ID NO: 11, the HCDR2 sequence of SEQ ID NO: 19, the HCDR3 sequence of SEQ ID NO: 27, the LCDR1 sequence of SEQ ID NO: 43, the LCDR2 sequence of SEQ ID NO: 51, and the LCDR3 sequence of SEQ ID NO: 59;
(6) an antibody comprising the VH sequence of SEQ ID NO: 3 and the VL sequence of SEQ ID NO: 35;
(7) an antibody comprising the HCDR1 sequence of SEQ ID NO: 12, the HCDR2 sequence of SEQ ID NO: 20, the HCDR3 sequence of SEQ ID NO: 28, the LCDR1 sequence of SEQ ID NO: 44, the LCDR2 sequence of SEQ ID NO: 52, and the LCDR3 sequence of SEQ ID NO: 60;
(8) an antibody comprising the VH sequence of SEQ ID NO: 4 and the VL sequence of SEQ ID NO: 36;
(9) an antibody comprising the HCDR1 sequence of SEQ ID NO: 13, the HCDR2 sequence of SEQ ID NO: 21, the HCDR3 sequence of SEQ ID NO: 29, the LCDR1 sequence of SEQ ID NO: 45, the LCDR2 sequence of SEQ ID NO: 53, and the LCDR3 sequence of SEQ ID NO: 61;
(10) an antibody comprising the VH sequence of SEQ ID NO: 5 and the VL sequence of SEQ ID NO: 37;
(11) an antibody comprising the HCDR1 sequence of SEQ ID NO: 14, the HCDR2 sequence of SEQ ID NO: 22, the HCDR3 sequence of SEQ ID NO: 30, the LCDR1 sequence of SEQ ID NO: 46, the LCDR2 sequence of SEQ ID NO: 54, and the LCDR3 sequence of SEQ ID NO: 62;
(12) an antibody comprising the VH sequence of SEQ ID NO: 6 and the VL sequence of SEQ ID NO: 38;
(13) an antibody comprising the HCDR1 sequence of SEQ ID NO: 16, the HCDR2 sequence of SEQ ID NO: 24, the HCDR3 sequence of SEQ ID NO: 32, the LCDR1 sequence of SEQ ID NO: 48, the LCDR2 sequence of SEQ ID NO: 56, and the LCDR3 sequence of SEQ ID NO: 64;
(14) an antibody comprising the VH sequence of SEQ ID NO: 8 and the VL sequence of SEQ ID NO: 40;
(15) an antibody that binds to the same HLA-DQ2.5 epitope and HLA-DQ8 epitope bound by the antibody of any one of (1) to (14);
(16) an antibody that competes for binding of HLA-DQ2.5 and HLA-DQ8 with the antibody of any one of (1) to (14).

8. A method for treating celiac disease, comprising administering to a patient a therapeutically effective amount of the antibody of any one of claims 1 to 7.

9. The antibody of any one of claims 1 to 7 for use in treating celiac disease.

10. A pharmaceutical composition for use in treating celiac disease, which comprises the antibody of any one of claims 1 to 7 and a pharmaceutically acceptable carrier.

11. A method of screening for an anti-HLA-DQ2.5/8 antibody, which comprises:
(a) testing whether an antibody has binding activity to HLA-DQ2.5 and HLA-DQ8; and selecting an antibody that has binding activity to HLA-DQ2.5 and HLA-DQ8;
(b) testing whether an antibody has a specific binding activity to HLA-DR or HLA-DP; and selecting an antibody that has substantially no specific binding activity to HLA-DR or HLA-DP;
(c) testing whether an antibody has a specific binding activity to a complex of the invariant chain and HLA-DQ2.5; and selecting an antibody that has substantially no specific binding activity to the complex of the invariant chain and HLA-DQ2.5.

12. The method of claim 11, which further comprises: testing whether an antibody has binding activity to HLA-DQ2.5 and HLA-DQ8 in the presence of a gluten peptide; and selecting an antibody that has binding activity to HLA-DQ2.5 and HLA-DQ8 in the presence of the gluten peptide.

13. The method of claim 11, which further comprises: testing whether an antibody has binding activity to monkey MHC-DQ; and selecting an antibody that has binding activity to monkey MHC-DQ.

14. The method of claim 11, which further comprises: testing whether an antibody has neutralizing activity against the binding between HLA-DQ2.5 and TCR and the binding between HLA-DQ8 and TCR; and selecting an antibody that has the neutralizing activity.

15. A method of preparing a pharmaceutical formulation for use in treating celiac disease, which comprises mixing a pharmaceutically acceptable carrier and the antibody selected by the method of claim 11.


SEQUENCE LISTING (81 sequences)

SEQ ID NO: 1 - protein, 123 aa, Oryctolagus cuniculus
QSLEESGGDLVKPGASLTLTCTASGFSFSSRYWMCWVRQAPGKGLEWIACIDAGSSGITYYASWANGRFTISKTSSTTVTLQMTSLTAADTATYFCARDAWSTTDGWNTFNLWGPGTLVTVSS

SEQ ID NO: 2 - protein, 120 aa, Oryctolagus cuniculus
QEQVVEYGGDLVQPEGSLTLTCKASGLDFSSTYYMCWVRQAPGKGLEWIGCIYGGSSDSTYYASWAKGRFTISKTSPTTVTLQMTSLTAADTATHFCATYDYGAVGYDLWGPGTLVTVSS

SEQ ID NO: 3 - protein, 118 aa, Oryctolagus cuniculus
QSVEESGGRLVTPGTPLTLTCTVSGFSLSSYYISWVRQAPGKGLEWIGIIYTDGVTDYPNWAKGRFTISKTSTTVDLKITSPTTEDTATYFCARDAASVYGCGYFDLWGPGTLVTVSS

SEQ ID NO: 4 - protein, 121 aa, Oryctolagus cuniculus
QQQLEESGGGLVKPGASLTLTCKGSGFSFTSGYWICWVRQAPGKGLEWIGCIYTGSSGSTYYASWAKGRFTISRSSTTVTLQMTSLTVADTATYFCAGDPSGASSGWDNLWGPGTLVTVSS

SEQ ID NO: 5 - protein, 121 aa, Oryctolagus cuniculus
QSLEESGGDLVKPGASLTLTCTASGFSFSSSYYMCWVRQAPGKGLEWIACIYTGSSGSTYYPSWAKGRFTISKTSSTTVTLQMTSLTAADTATYFCARVRAGSGYYYFNLWGPGTLVTVSS

SEQ ID NO: 6 - protein, 117 aa, Oryctolagus cuniculus
QSLEESGGRLVTPGTALTLTCTVSGFSLSSYAMGWVRQAPGKGLEYIGWTSPGDSAYYASWTKGRFTISRTSTTVELKITSPTTEDTATYFCARDAAYIGHWAFNLWGPGTLVTVSS

SEQ ID NO: 7 - skipped

SEQ ID NO: 8 - protein, 125 aa, Oryctolagus cuniculus
QSVEESGGRLVTPGTPLTLTCTVSGFSLSSYAMSWVRQAPGKGLEWIGVISYSGRTYDASWAKGRFTISKTSTTVDLKITSPTTEDTATYFCARVGDSYGYAYATVIFTYHFNLWGPGTLVTVSS

SEQ ID NO: 9 - protein, 6 aa, Artificial sequence
  DQN0016Hc HCDR1
SRYWMC

SEQ ID NO: 10 - protein, 6 aa, Artificial sequence
  DQN0089Hf HCDR1
STYYMC

SEQ ID NO: 11 - protein, 5 aa, Artificial sequence
  DQN0092Hh HCDR1
SYYIS

SEQ ID NO: 12 - protein, 6 aa, Artificial sequence
  DQN0100Ha HCDR1
SGYWIC

SEQ ID NO: 13 - protein, 6 aa, Artificial sequence
  DQN0157Hc HCDR1
SSYYMC

SEQ ID NO: 14 - protein, 5 aa, Artificial sequence
  DQN0139Hb HCDR1
SYAMG

SEQ ID NO: 15 - skipped

SEQ ID NO: 16 - protein, 5 aa, Artificial sequence
  DQN0177Ha HCDR1
SYAMS

SEQ ID NO: 17 - protein, 18 aa, Artificial sequence
  DQN0016Hc HCDR2
CIDAGSSGITYYASWANG

SEQ ID NO: 18 - protein, 18 aa, Artificial sequence
  DQN0089Hf HCDR2
CIYGGSSDSTYYASWAKG

SEQ ID NO: 19 - protein, 16 aa, Artificial sequence
  DQN0092Hh HCDR2
IIYTDGVTDYPNWAKG

SEQ ID NO: 20 - protein, 18 aa, Artificial sequence
  DQN0100Ha HCDR2
CIYTGSSGSTYYASWAKG

SEQ ID NO: 21 - protein, 18 aa, Artificial sequence
  DQN0157Hc HCDR2
CIYTGSSGSTYYPSWAKG

SEQ ID NO: 22 - protein, 16 aa, Artificial sequence
  DQN0139Hb HCDR2
WTSPGDSAYYASWTKG

SEQ ID NO: 23 - skipped

SEQ ID NO: 24 - protein, 16 aa, Artificial sequence
  DQN0177Ha HCDR2
VISYSGRTYDASWAKG

SEQ ID NO: 25 - protein, 14 aa, Artificial sequence
  DQN0016Hc HCDR3
DAWSTTDGWNTFNL

SEQ ID NO: 26 - protein, 10 aa, Artificial sequence
  DQN0089Hf HCDR3
YDYGAVGYDL

SEQ ID NO: 27 - protein, 13 aa, Artificial sequence
  DQN0092Hh HCDR3
DAASVYGCGYFDL

SEQ ID NO: 28 - protein, 12 aa, Artificial sequence
  DQN0100Ha HCDR3
DPSGASSGWDNL

SEQ ID NO: 29 - protein, 12 aa, Artificial sequence
  DQN0157Hc HCDR3
VRAGSGYYYFNL

SEQ ID NO: 30 - protein, 12 aa, Artificial sequence
  DQN0139Hb HCDR3
DAAYIGHWAFNL

SEQ ID NO: 31 - skipped

SEQ ID NO: 32 - protein, 20 aa, Artificial sequence
  DQN0177Ha HCDR3
VGDSYGYAYATVIFTYHFNL

SEQ ID NO: 33 - protein, 111 aa, Oryctolagus cuniculus
DVVMTQTPASVSAAVGGTVTIKCQASQSIGSNLNWYQQKPGQSPKLLIYSASTLASGVPSRFKGSGSGTEYTLTISNLECADAATYYCQSYYYTGSSAVNNFGGGTKVEIK

SEQ ID NO: 34 - protein, 111 aa, Oryctolagus cuniculus
EVVVTQTPASVEVAVGGTVTIKCQASQNISPYLSWYQQKPGQPPKLLIYKASTLASGVSSRFKGSGSGTQFTLTISDLECADAATYYCQNNYGVSINYGHTFGGGTKVEIK

SEQ ID NO: 35 - protein, 110 aa, Oryctolagus cuniculus
TFKLTQTPASVEAAVGGTVTIKCQASESNGNALAWYQQKPGQPPKLLIYRAFTLASGVPSRLKGSGSGTQFALTISDLECADAATYYCQTYYGSNTDFNAFGGGTKVEIK

SEQ ID NO: 36 - protein, 112 aa, Oryctolagus cuniculus
AFELTQTPSSVEAAVGGTVTIKCQASQTIVSWLAWYQQKPGQRPKLLILYASNLASGVSSRFRGSGSGTDYTLTISDLECDDAATYYCQSYYFSTSSPDMYAFGGGTKVEIK

SEQ ID NO: 37 - protein, 112 aa, Oryctolagus cuniculus
DIVMTQTPSSVSAAVGGTVTIKCQASQTIYSGLAWYQQKPGQPPKLLIYDASDLASGVPSRFSGSGSGTEFTLAISGVQCDDAATYYCHGDYYGGSGTFFWTFGGGTKVEIK

SEQ ID NO: 38 - protein, 110 aa, Oryctolagus cuniculus
DVVMTQTPASVSAPVGGTVTINCQASESIYSNLAWYQQKPGQPPKLLIYGASTLESGVPSRVKGSGYGTQFTLTISDLECADAATYYCQQYYGSSSTAFTFGGGTKVEIK

SEQ ID NO: 39 - skipped

SEQ ID NO: 40 - protein, 111 aa, Oryctolagus cuniculus
DPVLTQTPSSASEPVGGTVTIKCQASESISSSLAWYQQKPGQRPKLLIYDASKLASGVPSRFKGSGSGTEFTLTISGVQCDDAATYYCQHGLSSGSTNRCAFGGGTKVEIK

SEQ ID NO: 41 - protein, 11 aa, Artificial sequence
  DQN0016Lc LCDR1
QASQSIGSNLN

SEQ ID NO: 42 - protein, 11 aa, Artificial sequence
  DQN0089Lf LCDR1
QASQNISPYLS

SEQ ID NO: 43 - protein, 11 aa, Artificial sequence
  DQN0092Lh LCDR1
QASESNGNALA

SEQ ID NO: 44 - protein, 11 aa, Artificial sequence
  DQN0100La LCDR1
QASQTIVSWLA

SEQ ID NO: 45 - protein, 11 aa, Artificial sequence
  DQN0157Lc LCDR1
QASQTIYSGLA

SEQ ID NO: 46 - protein, 11 aa, Artificial sequence
  DQN0139Lb LCDR1
QASESIYSNLA

SEQ ID NO: 47 - skipped

SEQ ID NO: 48 - protein, 11 aa, Artificial sequence
  DQN0177La LCDR1
QASESISSSLA

SEQ ID NO: 49 - protein, 7 aa, Artificial sequence
  DQN0016Lc LCDR2
SASTLAS

SEQ ID NO: 50 - protein, 7 aa, Artificial sequence
  DQN0089Lf LCDR2
KASTLAS

SEQ ID NO: 51 - protein, 7 aa, Artificial sequence
  DQN0092Lh LCDR2
RAFTLAS

SEQ ID NO: 52 - protein, 7 aa, Artificial sequence
  DQN0100La LCDR2
YASNLAS

SEQ ID NO: 53 - protein, 7 aa, Artificial sequence
  DQN0157Lc LCDR2
DASDLAS

SEQ ID NO: 54 - protein, 7 aa, Artificial sequence
  DQN0139Lb LCDR2
GASTLES

SEQ ID NO: 55 - skipped

SEQ ID NO: 56 - protein, 7 aa, Artificial sequence
  DQN0177La LCDR2
DASKLAS

SEQ ID NO: 57 - protein, 13 aa, Artificial sequence
  DQN0016Lc LCDR3
QSYYYTGSSAVNN

SEQ ID NO: 58 - protein, 13 aa, Artificial sequence
  DQN0089Lf LCDR3
QNNYGVSINYGHT

SEQ ID NO: 59 - protein, 12 aa, Artificial sequence
  DQN0092Lh LCDR3
QTYYGSNTDFNA

SEQ ID NO: 60 - protein, 14 aa, Artificial sequence
  DQN0100La LCDR3
QSYYFSTSSPDMYA

SEQ ID NO: 61 - protein, 14 aa, Artificial sequence
  DQN0157Lc LCDR3
HGDYYGGSGTFFWT

SEQ ID NO: 62 - protein, 12 aa, Artificial sequence
  DQN0139Lb LCDR3
QQYYGSSSTAFT

SEQ ID NO: 63 - skipped

SEQ ID NO: 64 - protein, 13 aa, Artificial sequence
  DQN0177La LCDR3
QHGLSSGSTNRCA

SEQ ID NO: 65 - protein, 328 aa, Artificial sequence
  F1332m CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 66 - protein, 107 aa, Artificial sequence
  k0MC CL
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 67 - protein, 19 aa, Artificial sequence
  CAMPATH-1H signal sequence
MGWSCIILFLVATATGVHS

SEQ ID NO: 68 - protein, 4 aa, Artificial sequence
  GGGG linker
GGGG

SEQ ID NO: 69 - protein, 33 aa, Artificial sequence
  33mer gliadin peptide sequence
LQLQPFPQPELPYPQPELPYPQPELPYPQPQPF

SEQ ID NO: 70 - protein, 5 aa, Artificial sequence
  GGGGG linker
GGGGG

SEQ ID NO: 71 - protein, 12 aa, Artificial sequence
  SSADLVPRGGGG linker
SSADLVPRGGGG

SEQ ID NO: 72 - protein, 18 aa, Artificial sequence
  gliadin peptide sequence
QQYPSGEGSFQPSQENPQ

SEQ ID NO: 73 - protein, 13 aa, Artificial sequence
  SSADLVPRGGGGG linker
SSADLVPRGGGGG

SEQ ID NO: 74 - protein, 5 aa, Artificial sequence
  GGGGS linker
GGGGS

SEQ ID NO: 75 - protein, 18 aa, Artificial sequence
  CAMPATH-1H signal sequence
MGWSCIILFLVATATGVH

SEQ ID NO: 76 - protein, 18 aa, Artificial sequence
  signal sequence, rat serum albumin
MKWVTFLLLLFISGSAFS

SEQ ID NO: 77 - protein, 255 aa, Macaca fascicularis
MILNKALLLGALALTTVMSPCGGEDIVADHVASYGVNMYQSYGPSGQYTHEFDGDEEFYVDLERKQTVWQLPLFSKFRRFDPQGALRNIAVLQHNLNIVIKRSNSTAATNEVPEVTVFSKSPVMLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVFIIRGLRSIGASRHQGPL

SEQ ID NO: 78 - protein, 261 aa, Macaca fascicularis
MPRKKALRIPGGLWGTTVTLMLAMLSTPVAGGRDSPEDFVYQFKGLCYFTNGTERVRLVTRYIYNREEYVRFDSDWGEYRAVTPLGPPAAEYWNSQKDILERTRAELDTVCRHNYQSELRTTLQRRVEPTVTISPSRTEALNHHNLLVCSVTDFYPGQIKVRWFRNDQEETAGIVSTPLIRNGDWTFQILVMLEMTPQRGDVYTCHVEHPSLQSPITVEWRAQSESAQSKMLSGVGGFVLGLIFLGLGLIIHYRSQKGLLH

SEQ ID NO: 79 - protein, 255 aa, Macaca fascicularis
MILNKALMLGALVLTAVMSPCGGEDIVADHVASCGVNLYQTYGLSGQFTHEFDGDEQFYVDLRRKETAWRWPELSKFGGFDPQGALRNLAVLKHNLNIVTKRYNSTAATNEVPEVTVFSKSPVTLGHPNTLICLVDNIFPPVVNITWLSNGRSVTEGVSETSFLSKSDHSFFKISYLTFLPSADEIYDCRVEHWGLDEPLLKHWEPEIPAPMSELTETVVCALGLSVGLVGIVVGTVFIIQGLRSVGASRHQGPL

SEQ ID NO: 80 - protein, 261 aa, Macaca fascicularis
MSWKKALRIPSGLRVATVTLMLAMLSTPVAEGRDSPEDFVFQFKGLCYFTNGTERVRHVTRYIYNREEFLRFDSAVGEYRAVTPQGRSDAEHLNSQKDVLESTRAELDTVCMHNYEVTYRGILQRRVEPTVTISPSRTEALNHHNLLVCSVTDFYPGQIKVRWFRNNQEETVGVVSTPLIRNGDWTFQILVMLEMTPQRGDVYTCHVEHPSLQSPITVEWRAQSESAQSKMLSGIGGFVLGLIFFGLGLIIRQRSQKGLLH

SEQ ID NO: 81 - protein, 261 aa, Macaca fascicularis
MSWKKALRIPGGLRVATVTLMLAMLSTPVTEGRDSPEDFVYQFKGMCYFTNGTERVRHVTRYIYNREEYARFDSDWGEYRAVTPQGRPDAEYWNSQKDILESTRAELDTVCKHNYEVAFRGILQRRVEPTVTISPSRTEALNHHNLLVCSVTDFYPGQIKVRWFRNNQEETVGVVSTPLIRNGDWTFQILVMLEMTPQRGDVYTCHVEHPSLQSPITVEWRAQSESAQSKLLSGIGGLVLGLIFFGLGLIIRQRSQKGLPR